(19) United States
(12) Patent Application Publication
(10) Pub. No.: US 2022/0187184 A1
(43) Pub. Date: Jun. 16, 2022

(54) MICROFLUIDIC DEVICE FOR SINGLE CELL PROCESSING AND METHOD AND SYSTEM FOR SINGLE CELL BIOPHYSICAL PHENOTYPING USING THE MICROFLUIDIC DEVICE

(71) Applicant: Singapore University of Technology and Design, Singapore (SG)

(72) Inventors: Ye AL, Singapore (SG); Dahou YANG, Singapore (SG)

(21) Appl. No.: 17/436,101

(22) PCT Filed: Mar. 5, 2020
(86) PCT No.: PCT/SG2020/050105

§ 371 Date: Sep. 3, 2021

(30) Foreign Application Priority Data
Mar. 5, 2019 (SG) 10201901960P

(51) Int. Cl. G01N 15/10; B01L 3/00

(52) U.S. Cl. CPC G01N 15/1031; B01L 3/502715; B01L 2400/082; B01L 2300/0645; B01L 2300/0877; B01L 3/502776

(57) ABSTRACT

A microfluidic device includes a substrate; a plurality of electrode channels, including a first electrode channel, a second electrode channel, a third electrode channel and a fourth electrode channel, each containing an electrode material to form an electrode; and a plurality of fluidic channels, including a first fluidic channel and a second fluidic channel, each being configured to form a fluid pathway for allowing a fluid sample to flow through and at least one of the first and second fluidic channels including a cell manipulation portion, the cell manipulation portion including a plurality of constriction portions. The first and second electrode channels are each coupled to the first fluidic channel and the electrodes of the first and second electrode channels and the third and fourth electrode channels are each coupled to the second fluidic channel and the electrodes of the third and fourth electrode channels.

### CROSS-REFERENCE TO RELATED APPLICATION

This is a national phase application of PCT Application PCT/SG2020/050105, which was filed on Mar. 5, 2021, and which claims the benefit of priority of Singapore Patent Application No. 10201901960P, filed 5 Mar. 2019, the content of each of these being hereby incorporated by reference in their entirety for all purposes.

### TECHNICAL FIELD

The present invention generally relates to a microfluidic device for single cell processing, a method of manufacturing the microfluidic device, a method and a system for single cell biophysical phenotyping using the microfluidic device.

### BACKGROUND

Cellular biophysical properties (e.g., mechanical and electrical properties) are promising label-free biomarkers for classifying different cell types and studying their developmental stages. It has been proved that changes in cell deformability are related to several diseases, such as cancer, sepsis, and diabetes. For example, red blood cells infected with malaria parasites are much stiffer than normal red blood cells. Standard tools for studying the mechanical properties of single cells include atomic force microscopy, micropipette, and optical stretching. However, these techniques usually have very low throughput (e.g., less than 1 cell/min), as well as being labor-intensive and time-consuming.

Microfluidics-based technologies, for example hydrodynamic stretching-based techniques and constriction-based techniques, have previously been developed to characterize cell deformability at single cell level. In such techniques previously developed, high-speed camera setups (having high-speed imaging capability) are usually employed to visually capture the motion and shape change of single cells, and then the cell deformability is quantified by evaluating the elongation and compression of the single cell compared with its original circularity (hydrodynamic stretching), or the transit time of the single cell passing through a single-constriction channel (constriction-based). However, the high-speed camera setups are expensive, and also generate a huge amount of useless images without cell events that require an intricate image-processing algorithm to extract useful information.

Electrical impedance-based microfluidic devices employing a single-constriction channel have been previously disclosed for measuring the mechanical and electrical properties of single cells (e.g., characterizing the deformability and electrical impedance of single cells). A common configuration of such devices is that an electrode is positioned at an input side and another electrode is positioned at an output side of the single constriction. In this regard, an AC voltage is applied to the electrode at the input side and an electrical current is measured from the other electrode at the output side of the single constriction. When a single cell flows through the single constriction, it temporally blocks the electrical fields inside the single constriction, giving rise to an increase in electrical impedance across the two electrodes. The transit time of a single cell through the single constriction may then be calculated by extracting the time duration between the impedance changes at the entrance (input) and exit (output) of the single constriction. The transit time calculated may be used as an indicator of cell deformability. However, such existing electrical impedance-based microfluidic devices usually employ a PDMS (polydimethylsiloxane) layer containing microfluidic channels and an electrode substrate. In this regard, the fabrication of the electrode substrate (microfabricated coplanar electrodes) involves multiple steps, including photolithography, metal deposition and lift-off. These electrode fabrication steps often require complex and sophisticated equipment (e.g., e-beam evaporator or sputtering machine for metal deposition), which is generally expensive, relatively slow and not readily accessible. In addition, the bonding of electrode patterns and PDMS channels also requires precise alignment, making this process challenging and time-consuming. Furthermore, electrical impedance measurements obtained from such existing electrical impedance-based microfluidic devices may also be inferior.

A need therefore exists to provide a microfluidic device for single cell processing and method and system for single cell biophysical phenotyping using the microfluidic device that seek to overcome, or at least ameliorate, one or more of the deficiencies of conventional microfluidic devices and conventional method and system for single cell biophysical phenotyping. It is against this background that the present invention has been developed.

### SUMMARY

According to a first aspect of the present invention, there is provided a microfluidic device for single cell processing, the microfluidic device comprising:

a substrate;

a plurality of electrode channels, comprising a first electrode channel, a second electrode channel, a third electrode channel and a fourth electrode channel, provided in the substrate, each of the plurality of electrode channels containing an electrode material to form an electrode; and

a plurality of fluidic channels, comprising a first fluidic channel and a second fluidic channel, provided in the substrate, each of the plurality of fluidic channels being configured to form a fluid pathway for allowing a fluid sample to flow through and at least one of the first and second fluidic channels comprising a cell manipulation portion, the cell manipulation portion comprising a plurality of constriction portions, wherein

the first and second electrode channels are each coupled to the first fluidic channel and the electrodes of the first and second electrode channels are configured to measure an electrical impedance therebetween via the first fluidic channel, and

the third and fourth electrode channels are each coupled to the second fluidic channel and the electrodes of the third and fourth electrode channels are configured to measure an electrical impedance therebetween via the second fluidic channel.

According to a second aspect of the present invention, there is provided a method of manufacturing a microfluidic device for single cell processing, the method comprising:

providing a substrate;

forming a plurality of electrode channels, comprising a first electrode channel, a second electrode channel, a third electrode channel and a fourth electrode channel, in the substrate, each of the plurality of electrode channels containing an electrode material to form an electrode; and

forming a plurality of fluidic channels, comprising a first fluidic channel and a second fluidic channel, in the substrate, each of the plurality of fluidic channels being configured to form a fluid pathway for allowing a fluid sample to flow through and at least one of the first and second fluidic channels comprising a cell manipulation portion, the cell manipulation portion comprising a plurality of constriction portions, wherein

the first and second electrode channels are each coupled to the first fluidic channel and the electrodes of the first and second electrode channels are configured to measure an electrical impedance therebetween via the first fluidic channel, and

the third and fourth electrode channels are each coupled to the second fluidic channel and the electrodes of the third and fourth electrode channels are configured to measure an electrical impedance therebetween via the second fluidic channel.

According to a third aspect of the present invention, there is provided a method of single cell biophysical phenotyping using the microfluidic device for single cell processing as described according to the above-mentioned first aspect, the method comprising:

obtaining a first impedance measurement based on the electrodes of the first and second electrode channels with the first fluidic channel having a fluid sample flowing therein;

obtaining a second impedance measurement based on the electrodes of the third and fourth electrode channels with the second fluidic channel having a fluid sample flowing therein;

obtaining a differential impedance measurement based on the first impedance measurement and the second impedance measurement, the differential impedance measurement comprising a differential impedance signal; and

determining one or more biophysical properties of a single cell in the fluid sample that flowed in one of the first and second fluidic channels comprising the cell manipulation portion based on the differential impedance signal.

According to a fourth aspect of the present invention, there is provided a system for single cell biophysical phenotyping, the system comprising:

the microfluidic device for single cell processing as described according to the above-mentioned first aspect; and

a computing system comprising:
a memory; and
at least one processor communicatively coupled to the memory and the microfluidic device, and configured to:
obtain a first impedance measurement based on the electrodes of the first and second electrode channels with the first fluidic channel having a fluid sample flowing therein;
obtain a second impedance measurement based on the electrodes of the third and fourth electrode channels with the second fluidic channel having a fluid sample flowing therein;
obtain a differential impedance measurement based on the first impedance measurement and the second impedance measurement, the differential impedance measurement comprising a differential impedance signal; and
determine one or more biophysical properties of a single cell in the fluid sample that flowed in one of the first and second fluidic channels comprising the cell manipulation portion based on the differential impedance signal.

### BRIEF DESCRIPTION OF THE DRAWINGS

Embodiments of the present invention will be better understood and readily apparent to one of ordinary skill in the art from the following written description, by way of example only, and in conjunction with the drawings, in which:

FIG. 1A depicts a schematic drawing of a microfluidic device for single cell processing, according to various embodiments of the present invention;

FIG. 1B depicts a schematic drawing of a microfluidic device for single cell processing, according to various embodiments of the present invention, which is the same as the microfluidic device shown in FIG. 1A, except that the first and second fluidic channels each comprises a respective cell manipulation portion;

FIG. 2 depicts a schematic flow diagram of a method of manufacturing a microfluidic device for single cell processing, according to various embodiments of the present invention;

FIG. 3 depicts a schematic flow diagram of a method of single cell biophysical phenotyping using a microfluidic device for single cell processing, according to various embodiments;

FIG. 4 depicts a schematic drawing of a system for single cell biophysical phenotyping, according to various embodiments of the present invention;

FIG. 5 depicts a schematic block diagram of an exemplary computer system in which the system for single cell biophysical phenotyping, according to various embodiments of the present invention may be realized or implemented;

FIG. 6A depicts a top view of an example microfluidic device fabricated for single cell processing, according various example embodiments of the present invention;

FIG. 6B depicts an enlarged 3D schematic of the dotted boxed section of the microfluidic device shown in FIG. 6A, along with illustrative single cells flowing;

FIG. 6C depicts a schematic drawing of an example configuration of two fluidic channels, according to various example embodiments of the present invention;

FIG. 7A depicts a microscopic image of a section of two fluidic channels including the cell manipulation portions, according to various example embodiments of the present invention, along with notations indicating the electrical measurement setup;

FIG. 7B depicts two example scenarios, namely, a first scenario of a single cell flowing through an upper or first fluidic channel and a second scenario of a second cell flowing through a lower or second fluidic channel, according to various example embodiments of the present invention, along with the corresponding differential electrical signal profiles obtained;

FIG. 8 depicts an image of a microfluidic device fabricated, according to various example embodiments of the present invention;

FIG. 9 depicts a plot showing cell size distributions of normal MCF-7, CB-MCF-7 and NEM-MCF-7, according to various example embodiments of the present invention;

FIG. 10 depicts a schematic drawing illustrating an example back propagation neural network configured for classifying cell types based on biophysical properties measured, according to various example embodiments of the present invention;

FIG. 11A depicts an impedance signal of MCF-7 cells obtained passing through the constriction regions in the first and second fluidic channels, measured from one single experiment, according to various example embodiments of the present invention;

FIG. 11B depicts an enlarged section of the impedance signal shown in FIG. 11A, which corresponds to an event where one cell transits through the first fluidic channel, followed by another cell passing through the second fluidic channel, according to various example embodiments of the present invention;

FIG. 11C depicts box charts illustrating the total transit time for cells to pass through the four constrictions for three cell populations in an experiment performed, according to various example embodiments of the present invention;

FIG. 11D depicts box charts illustrating the average electrical impedance of cells passing through four successive constrictions for three cell populations in an experiment performed, according to various example embodiments of the present invention;

FIGS. 12A, 12B and 12C depict box charts illustrating the transit time of cells passing through each of the four successive constrictions for three cell populations, respectively, in an experiment performed, according to various example embodiments of the present invention;

FIGS. 13A, 13B and 13C depict box charts illustrating the electrical impedance of cells passing through each of the four successive constrictions for three cell populations, respectively, in an experiment performed, according to various example embodiments of the present invention;

FIG. 14A depicts box charts illustrating the relaxation index, namely, the ratio of the transit time of a single cell transiting through the fourth constriction to the transit time of the cell transiting through the first constriction, in an experiment performed, according to various example embodiments of the present invention;

FIG. 14B depicts a 3D scatterplot of electrical impedance vs total transit time vs relaxation index, according to various example embodiments of the present invention;

FIG. 15A depicts confusion matrices of the training, validation and test group without normalization, according to various example embodiments of the present invention;

FIG. 15B depicts confusion matrices of the training, validation and test group with normalization, according to various example embodiments of the present invention;

FIG. 15C depicts a table showing a summary of classification accuracy of different cell types, according to various example embodiments of the present invention;

FIG. 15D depicts a table showing a summary of sensitivity and specificity of cell type classification, according to various example embodiments of the present invention;

FIG. 16A depicts a flow cytometric scatter plot of the mixed MCF-7 and NEM-MCF-7 cells, according to various example embodiments of the present invention;

FIGS. 16B and 16C depict a 2D scatter plot and a 3D scatter plot, respectively, of the mixed MCF-7 and NEM-MCF-7 cells, according to various example embodiments of the present invention;

FIGS. 17A and 17B show the results of the passage time and electrical impedance, respectively, of cells passing through the constriction, according to various example embodiments of the present invention; and

FIG. 17C depicts a scatter plot the use of the combination of passage time and electrical impedance for effectively distinguish different cell populations, according to various example embodiments of the present invention.

### DETAILED DESCRIPTION

Embodiments of the present invention provide a microfluidic device for single cell processing, a method of manufacturing the microfluidic device, a method and a system for single cell biophysical phenotyping using the microfluidic device.

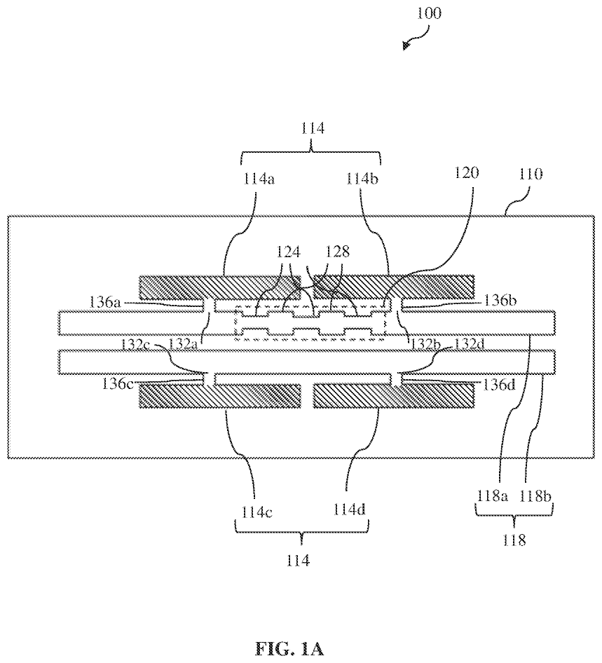

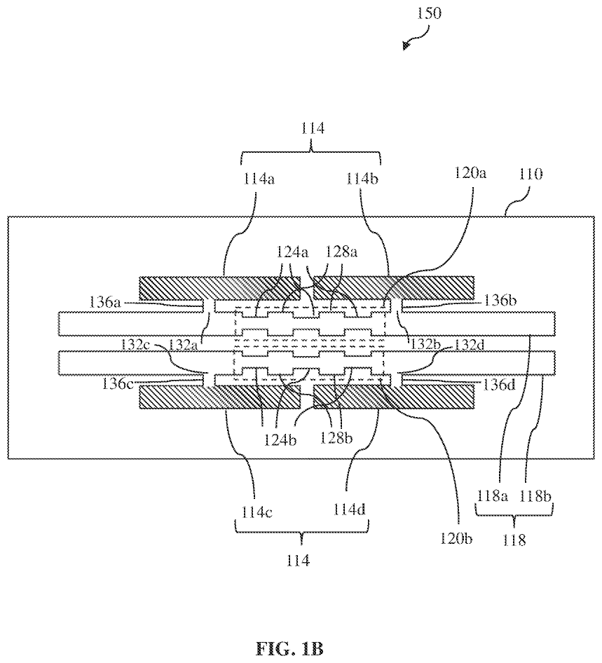

FIG. 1A depicts a schematic drawing of a microfluidic device 100 for single cell processing according to various embodiments of the present invention. The microfluidic device 100 comprises: a substrate 110; a plurality of electrode channels 114, comprising a first electrode channel 114a, a second electrode channel 114b, a third electrode channel 114c and a fourth electrode channel 114d, provided in the substrate 110, each of the plurality of electrode channels 114 containing an electrode material to form an electrode; and a plurality of fluidic channels 118, comprising a first fluidic channel 118a and a second fluidic channel 118b, provided in the substrate 110, each of the plurality of fluidic channels 118 being configured to form a fluid pathway for allowing a fluid sample to flow through and at least one of the first and second fluidic channels 118a/118b comprising a cell manipulation portion 120, the cell manipulation portion 120 comprising a plurality of constriction portions 120. The first and second electrode channels 114a/114b are each coupled to the first fluidic channel 118a and the electrodes (e.g., which may be referred to herein as first and second electrodes) of the first and second electrode channels 114a/114b are configured to measure an electrical impedance therebetween via the first fluidic channel 118a. The third and fourth electrode channels 114c/114d are each coupled to the second fluidic channel 118b and the electrodes (e.g., which may be referred to herein as third and fourth electrodes) of the third and fourth electrode channels 114c/114d are configured to measure an electrical impedance therebetween via the second fluidic channel 118b.

It can be understood by a person skilled in the art that for illustration purpose only and without limitation, FIG. 1A illustrates an example configuration (e.g., first example configuration) of the microfluidic device 100 whereby the first fluidic channel 118a comprises the cell manipulation portion 120. That is, the first fluidic channel 118a is the above-mentioned one of the first and second fluidic channels 118a/118b comprising the cell manipulation portion 120. It will be appreciated by a person skilled in the art that the microfluidic device 100 is not limited to the first fluidic channel 118a comprising the cell manipulation portion 120, and in another example configuration (e.g., second example configuration (not shown)), the second fluidic channel 118b may instead comprise the cell manipulation portion 120. That is, the second fluidic channel 118b is the above-mentioned one of the first and second fluidic channels 118a/118b comprising the cell manipulation portion 120. Furthermore, in yet another example configuration (e.g., third example configuration), the first and second fluidic channels 118a/118b may each comprise a respective cell manipulation portion, such as shown in FIG. 1B.

FIG. 1B depicts a schematic drawing of a microfluidic device 150 for single cell processing according to various embodiments of the present invention, which is the same as the microfluidic device 100, except that the first and second fluidic channels 118a/118b each comprises a respective cell manipulation portion 120a/120b, the cell manipulation portion 120a/120b comprising a plurality of constriction portions 124a/124b. As will be described later below, the cell manipulation portion 120a/120b may further comprise a plurality of relaxation portions 128a/128b.

For the sake of clarity and conciseness, unless stated otherwise, various embodiments of the present invention will be described hereinafter with reference to the microfluidic device 100 having an example configuration as shown in FIG. 1A (i.e., the first example configuration). It will be appreciated by a person skilled in the art that various features and associated advantages described with reference to the first example configuration may similarly, equivalently or correspondingly apply to the second and third example configurations, and thus need not be explicitly stated or repeated for clarity and conciseness.

The above-described configurations of the microfluidic device 100 for single cell processing advantageously provide a number of advantages, such as but not limited to, an improved electrical impedance measurement, an improved (e.g., high) throughput (e.g., number of cells processed per minute) and an improved (e.g., simpler) fabrication process, as well as enabling multiple types of biophysical properties of single cells to be determined.

In relation to the improved electrical impedance measurement, for example, the microfluidic device 100 is advantageously configured to include at least a pair of fluidic channels (e.g., the first fluidic channel 118a and the second fluidic channel 118b), at least a pair of electrodes (e.g., the first and second electrodes) configured to measure an electrical impedance therebetween via one of the pair of fluidic channels (e.g., the first fluidic channel 118a) and at least a pair of electrodes (e.g., the third and fourth electrodes) configured to measure an electrical impedance therebetween via the other one of the pair of fluidic channels (e.g., the second fluidic channel 118b), such that a differential electrical impedance measurement with respect to the pair of fluidic channels can be obtained, resulting in an improved electrical impedance measurement. In this regard, during one or more time periods, a pair of electrodes (e.g., the first and second electrodes) may measure an electrical impedance therebetween via the corresponding fluidic channel comprising the cell manipulation portion 120 (e.g., the first fluidic channel 118a) having only one single cell flowing therethrough, that is, only one single cell in the fluid sample flowing therethough (at least over a length of the corresponding fluidic channel between the two portions (coupling portions) of the corresponding fluidic channel coupled to the pair of electrodes) and another pair of electrodes (e.g., the third and fourth electrodes) may measure an electrical impedance therebetween via the corresponding fluidic channel (e.g., the second fluidic channel 118b) having no cell flowing through therebetween, that is, no cell in the fluid sample flowing therethrough (at least over a length of the corresponding fluidic channel between the two portions (coupling portions) of the corresponding fluidic channel coupled to the pair of electrodes). Accordingly, in such one or more time periods, the electrical impedance measured between the pair of electrodes via the fluidic channel having only one single cell flowing therethrough may thus correspond to that of the fluid sample including the single cell (which may be referred to as a signal electrical impedance measurement), and the electrical impedance measured between the pair of electrodes via the fluidic channel having no cell flowing therethrough may thus correspond to that of the fluid sample only (which may be referred to as a reference electrical impedance measurement). Accordingly, a differential electrical impedance measurement may be obtained based the signal electrical impedance measurement and the reference electrical impedance measurement, such as a difference between the signal electrical impedance measurement and the reference electrical impedance measurement. For example, such a differential electrical impedance measurement may cancel out common mode drifts caused by electrode properties or surrounding environment changes, thereby improving the measurement signal quality (e.g., improved signal-to-noise ratio).

In relation to the improved throughput, for example, the microfluidic device 100 is advantageously configured to process single cells, individually, based on electrical impedance measurement of the single cell flowing through the cell manipulation portion 120 including the plurality of constriction portions 124. As a result, one or more biophysical properties of the single cell (including multiple biophysical properties simultaneously) can be determined based on the electrical impedance measurement in relation to the single cell flowing through the plurality of constriction portions 124 in an accurate and efficient manner (e.g., without requiring complex image analysis), thus resulting in an improved throughput.

In relation to the improved fabrication process, for example, the microfluidic device 100 is advantageously configured to include a plurality of electrode channels 114, thereby enabling an electrode material to be introduced into each of plurality of electrode channels 114 to form a corresponding electrode therein. Accordingly, such a plurality of electrode channels 114 can be formed in the same or similar manner as the plurality of fluidic channels 118 in the microfluidic device 100, such as fabricated simultaneously via a single-step soft-lithography (e.g., without metal deposition and lift-off), thereby improving (e.g., simplifying fabrication process). Furthermore, as the electrode material may be introduced (in a liquid form) into each electrode channel 114, the electrode formed in the electrode channel 114 (in a solid form after the electrode material introduced solidifies) has a configuration that automatically conforms to the configuration of the electrode channel 114. In this regard, by fabricating the plurality of electrode channels 114 (at least a portion thereof) so as to be aligned with the plurality of fluidic channels 118, the electrodes formed in the plurality of electrode channels 114 (at least a portion thereof) are advantageously self-aligned with the plurality of fluidic channels 118, thus further improving (e.g., further simplifying fabrication process).

It will be appreciated by a person skilled in the art that the fluidic channels 118a/118b shown in FIG. 1A (and similarly in FIG. 1B) may only illustrate a portion of the fluidic channels 118a/118b, e.g., a linear portion of the fluidic channels 118a/118b. For example, such a linear portion of the fluidic channel 118a includes the cell manipulation portion 120. In other words, it will be appreciated that each fluidic channel 118a/118b may include other portions (not shown in FIG. 1A) as desired or as appropriate, such as but not limited to, a serpentine-shaped portion, an input portion (in fluid communication with a fluid inlet of the microfluidic device 100) and an output portion (in fluid communication with a fluid outlet of the microfluidic device 100). For example, it will be appreciated that an input portion may be provided for receiving a fluid sample and an output portion may be provided for outputting the fluid sample after flowing through the fluidic channel 118a/118b.

In various embodiments, each of the plurality of fluidic channels 118 may be coupled to (in fluid communication with) a flow splitter (not shown in FIG. 1A), for example, via the respective input portion. In this regard, the flow splitter may have an input portion configured to receive an input fluid sample from a fluid inlet of the microfluidic device 100 and a plurality of output portions configured to output a plurality of fluid samples (divided from the input fluid sample) to the plurality of fluidic channels 118, respectively, coupled thereto. With this configuration, the fluid sample flowing through each of the plurality of fluidic channels 118 may be from the same input fluid sample. It will be appreciated that the plurality of fluidic channels 118, the flow splitter, the fluid inlet, the fluid outlet and so on may form an integral or interconnected fluidic channel network.

In various embodiments, the plurality of fluidic channels 118 may be coupled to (in fluid communication with) a plurality of fluid inlets of the microfluidic device 100, respectively, for example, via respective input portions, for receiving a plurality of input fluid samples, respectively. With this configuration, the plurality of fluid samples flowing through the plurality of fluidic channels 118 may be the plurality of input fluid samples, respectively. For example, in the case of two fluidic channels, a first fluidic channel (comprising a cell manipulation portion 120) may be configured to receive a first input fluid sample comprising single cells desired to be analyzed and a second fluidic channel may be configured to receive a second input fluid sample without any cell. The second input fluid sample may be the same as the first fluid sample except that the second fluid sample is without any cell. The first fluidic channel may then function as a signal channel and the second fluidic channel may then function as a reference channel.

In various embodiments, the cell manipulation portion 120 further comprises a plurality of relaxation portions 128. In various embodiments, the plurality of relaxation portions 128 and the plurality of constriction portions 124 in the cell manipulation portion 120 are arranged alternately along the fluidic channel 118. For example, as shown in FIG. 1A, with respect to the first fluidic channel 118a, the plurality of relaxation portions 128 and the plurality of constriction portions 124 in the cell manipulation portion 120 are arranged alternately along the fluidic channel 118a.

In various embodiments, in the cell manipulation portion 120, each adjacent pair of constriction portions of the plurality of constriction portions 124 is interspaced by a corresponding relaxation portion of the plurality of relaxation portions 128. For example, as shown in FIG. 1A, with respect to the first fluidic channel 118a, each adjacent pair of constriction portions of the plurality of constriction portions 124 is interspaced by a corresponding relaxation portion of the plurality of relaxation portions 128.

In various embodiments, the above-mentioned at least one of the first and second fluidic channels 118 (e.g., the first fluidic channel 118a as shown in FIG. 1A) comprises a linear portion (e.g., generally straight), the linear portion comprising the cell manipulation portion 120. As explained hereinbefore, with respect to the fluidic channels 118, FIG. 1A may only illustrate a linear portion of the fluidic channels 118. For example, as shown in FIG. 1A, the first fluidic channel 118a comprises a linear portion, the linear portion comprising the cell manipulation portion 120.

In various embodiments, in the cell manipulation portion 120, each of the plurality of constriction portions 124 has a cross-sectional dimension (e.g., cross-sectional width) (or cross-sectional area) that is less than a cross-sectional dimension (or cross-sectional area) of a non-cell manipulation portion of the fluidic channel 118a. In this regard, the fluidic channel 118a, except the cell manipulation portion 120, that is, the non-cell manipulation portion, may be configured (e.g., designed or sized) to allow single cells (e.g., desired to be processed or analyzed, which may thus be referred to as desired single cells) to individually flow (i.e., one after another along the fluidic channel) through unhindered or unobstructed (e.g., without exerting any compressive force thereto to cause deformation). Accordingly, the non-cell manipulation portion may be configured to have a cross-sectional dimension (or cross-sectional area) that is larger than a cross-sectional dimension (or cross-sectional area) of the desired single cell to allow the desired single cells to individually flow therethrough unhindered, such as but not limited to about 200% to 500% larger. On the other hand, each of the plurality of constriction portions 124 is configured to manipulate (e.g., deform) each single cell, and is thus configured to have a cross-sectional dimension (or cross-sectional area) that is less than a cross-sectional dimension (or cross-sectional area) of a non-cell manipulation portion. In particular, each of the plurality of constriction portions 124a is configured (e.g., designed or sized) to have a cross-sectional dimension (or cross-sectional area) that is smaller than a cross-sectional dimension (or cross-sectional area) of the desired single cell, such as but not limited to about 20% to 180% smaller, to exert compressive pressure on the desired single cells as they individually flow through so as to deform the desired single cell.

In various embodiments, each of the plurality of relaxation portions 128 has a cross-sectional dimension (e.g., cross-sectional width) (or cross-sectional area) that is substantially equal to or greater than the cross-sectional dimension (or cross-sectional area) of the non-cell manipulation portion of the fluidic channel 118a. In this regard, contrary to the plurality of constriction portions 124, each of the plurality of relaxation portions 128 is configured (e.g., designed or sized) to allow the desired single cells to individually flow (i.e., one after another along the fluidic channel) through unhindered or unobstructed (e.g., without exerting any compressive force thereto to cause deformation). Accordingly, each of the plurality of relaxation portions 128 may be configured to have a cross-sectional dimension (or cross-sectional area) that is equal to or greater than the cross-sectional dimension of the non-cell manipulation portion of the fluidic channel 118a.

Accordingly, in various embodiments, each of the plurality of constriction portions 124 is configured to compress a single cell flowing therethrough, and each of the plurality of relaxation portions 128 is configured to decompress (or relax, such as by being configured to allow the desired single cells to individually flow through unhindered as described hereinbefore) the single cell flowing therethrough. In this regard, as described hereinbefore, the single cell has a size that the microfluidic device 100 is configured to process (e.g., desired to be processed or analyzed). For example, single cell sizes may range from about 1 μm to about 50 μm depending on the type of cells. By way of an example only and without limitation, the non-cell manipulation portion may be configured to have cross-sectional dimensions of about 30 μm to about 100 μm in width and about 10 μm to about 50 μm in height and each constriction portion may be configured to have cross-sectional dimensions of about 1 μm to about 30 μm in width and about 2 μm to about 20 μm in height for processing single cells having sizes in a range of about 1 μm to about 50 μm.

In various embodiments, each of the plurality of constriction portions 124 has an elongated shape extending in a direction of the fluid pathway. In various embodiments, each of the plurality of constriction portions 124 has dimensions which are the same. In this regard, for each of the plurality of constriction portions 124, a length of the constriction portion 124 corresponds to a time period which the single cell will be manipulated (e.g., deformed) by the constriction portion 124 as the single cell flows through, which also corresponds to a time period that the electrical impedance measured through the cell manipulation portion 120 correspondingly change (increase) due to the presence of the single cell in the constriction portion 124 affecting the electrical field lines in the constriction portion 124. A transit time of the single cell flowing through the constriction portion 124 may then be determined based on such an electrical impedance change. Accordingly, in various embodiments, each constriction portion 124 may be configured to have a length (in a direction of the length of the channel, that is, a longitudinal direction) based on a desired time period for the constriction portion 124 to manipulate to single cell flowing therethrough. For example, a shorter length may lead to a higher throughput but lower electrical impedance signal resolution (e.g., shorter time period that the electrical impedance change), whereas a longer length may lead to a higher electrical impedance signal resolution but a lower throughput (since the single cell has to travel more for processing). By way of an example only and without limitation, continuing from the above example range of cell sizes, each constriction portion 124 may have a length of about 10 μm to about 100 μm, and each relaxation portion 128 may have a length of about 10 μm to about 100 μm.

In various embodiments, the first and second electrode channels 114a/114b are coupled to the first fluidic channel 118a at coupling portions, including a first coupling portion 132a and a second coupling portion 132b, of the first fluidic channel 118a, respectively. In various embodiments, the third and fourth electrode channels 114c/114d are coupled to the second fluidic channel 118b at coupling portions, including a third coupling portion 132c and a fourth coupling portion 132d, of the second fluidic channel 118b, respectively. In various embodiments, the cell manipulation portion 120 of the above-mentioned at least one the first and second fluidic channels 118 is provided between the coupling portions of the corresponding fluidic channel such that the electrodes of the corresponding electrode channels are configured to measure the electrical impedance therebetween via the cell manipulation portion 120 of the corresponding fluidic channel. For example, in the example configuration of FIG. 1A, the cell manipulation portion 120 of the first fluidic channel 118a is provided between the coupling portions (i.e., the first and second coupling portions 132a/132b) of the first fluidic channel 118a such that the electrodes (first and second electrodes) of the corresponding electrode channels 114a/114b are configured to measure the electrical impedance therebetween via the cell manipulation portion 120 of the corresponding fluidic channel 118a. In the example configuration of FIG. 1A, the coupling portions (i.e., the third and fourth coupling portions 132c/132d) of the second fluidic channel 118b may be arranged or positioned along the second fluidic channel 118b so as to respectively correspond to the positions of the coupling portions (i.e., the first and second coupling portions 132a/132b) of the first fluidic channel 118a along the first fluidic channels 118a, that is, at corresponding or same positions, such that the coupling portions 132c/132d are spaced apart from each other by the same distance as the coupling portions 132a/132b. For example, this is so that the electrical impedance measured between the first and second electrodes and the electrical impedance measured between the third and fourth electrodes are via the same or similar electrical conduction path length.

For example, in the example configuration of FIG. 1B, the first cell manipulation portion 120a of the first fluidic channel 118a is provided between the first and second coupling portions 132a/132b of the first fluidic channel 118a such that the electrodes of the first and second electrode channels 114a/114b are configured to measure the electrical impedance therebetween via the first cell manipulation portion 120a of the first fluidic channel 118a. The second cell manipulation portion 120b of the second fluidic channel 118b is provided between the third and fourth coupling portions 132c/132d of the second fluidic channel 118b such that the electrodes of the third and fourth electrode channels 132c/132d are configured to measure the electrical impedance therebetween via the second cell manipulation portion 120b of the second fluidic channel 118b.

In various embodiments, the first and second electrode channels 114a/114b are coupled to the first fluidic channel 118a (to the first and second coupling portions 132a/132b thereof, respectively) via a first coupling channel 136a and a second coupling channel 136b, respectively. Each of the first and second coupling channels 136a/136b is configured to form a fluid pathway for allowing the fluid sample to flow so as to expose the electrodes of the first and second electrode channels 114a/114b for direct contact with the fluid sample when the fluid sample is being flowed in the first fluidic channel 118a. In various embodiments, the third and fourth electrode channels 114c/114d are coupled to the second fluidic channel 118b (to the third and fourth coupling portions 132c/132d thereof, respectively) via a third coupling channel 136c and a fourth coupling channel 136d, respectively. Similarly, each of the third and fourth coupling channels 136c/136d is configured to form a fluid pathway for allowing the fluid sample to flow so as to expose the electrodes of the third and fourth electrode channels 114c/114d for direct contact with the fluid sample when the fluid sample is being flowed in the second fluidic channel 118b. Accordingly, when a fluid sample is flowing through the first fluidic channel 118a, an electrical conduction path may be formed between the electrodes of the first and second electrode channels 114a/114b through the cell manipulation portion 120 for measuring an electrical impedance therebetween. Similarly, when a fluid sample is flowing through the second fluidic channel 118b, an electrical conduction path may be formed between the electrodes of the third and fourth electrode channels 114c/114d for measuring an electrical impedance therebetween.

In various embodiments, the cell manipulation portion 120 comprises three or more constriction portions 124. Based on this configuration, various embodiments advantageously determine multiple biophysical properties of a single cell based on the electrical impedance measurement in relation to the single cell flowing through the cell manipulation portion 120. In various embodiments, in the example configuration of FIG. 1B, the cell manipulation portion 120a of the first fluidic channel 118a and the cell manipulation portion 120b of the second fluidic channel 118b have the same configuration (e.g., identical dimensions).

In various embodiments, the first fluidic channel 118a and the second fluidic channel 118b are parallel to each other. In various embodiments, the first and second electrode channels 114a/114b each comprises an aligned portion configured to be adjacent the first fluidic channel 118a and to be aligned with the first fluidic channel 118a so as to extend in a parallel manner thereto. Similarly, in various embodiments, the third and fourth electrode channels 114c/114d each comprises an aligned portion configured to be adjacent the second fluidic channel 118b and to be aligned with the second fluidic channel 118b so as to extend in a parallel manner thereto. Similar to the fluidic channels 118a/11b shown in FIG. 1A, for each of the plurality of electrode channels 114 shown in FIG. 1A may only illustrate a portion of the electrode channel, e.g., the above-mentioned aligned portion of the electrode channel 114, In other words, it will be appreciated that each electrode channel may include other portions (not shown in FIG. 1A) as desired or as appropriate, such as but not limited to, an input portion coupled to an inlet of the microfluidic device for receiving an electrode material in a liquid form into the electrode channel to form an electrode therein. In this regard, as described hereinbefore, as the electrode material may be introduced in a liquid form into each electrode channel, the electrode formed in the electrode channel when solidified has a configuration that automatically conforms to the configuration of the electrode channel. Accordingly, as the plurality of electrode channels 114 (i.e., the aligned portions thereof) are fabricated so as to be aligned with the plurality of fluidic channels 118, respectively, the electrodes formed in the plurality of electrode channels 114 are advantageously self-aligned with the plurality of fluidic channels 118, respectively. In this regard, such an aligned portion advantageously facilitates the generation of uniform electric field along the height of the corresponding portion of the fluidic channel.

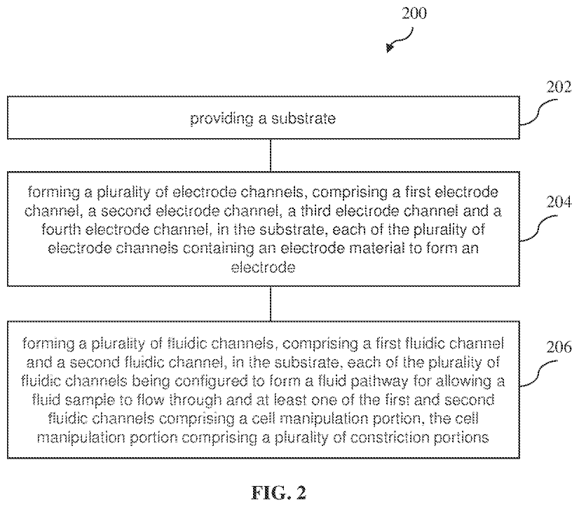

FIG. 2 depicts a schematic flow diagram of a method 200 of manufacturing a microfluidic device for single cell processing, such as the microfluidic device 100 or 150 as described herein with reference to FIG. 1A or FIG. 1B. The method 200 comprises: providing (at 202) a substrate; forming (at 204) a plurality of electrode channels, comprising a first electrode channel, a second electrode channel, a third electrode channel and a fourth electrode channel, in the substrate, each of the plurality of electrode channels containing an electrode material to form an electrode; and forming (at 206) a plurality of fluidic channels, comprising a first fluidic channel and a second fluidic channel, in the substrate, each of the plurality of fluidic channels being configured to form a fluid pathway for allowing a fluid sample to flow through and at least one of the first and second fluidic channels comprising a cell manipulation portion, the cell manipulation portion comprising a plurality of constriction portions. The first and second electrode channels are each coupled to the first fluidic channel and the electrodes of the first and second electrode channels are configured to measure an electrical impedance therebetween via the first fluidic channel. The third and fourth electrode channels are each coupled to the second fluidic channel and the electrodes of the third and fourth electrode channels are configured to measure an electrical impedance therebetween via the second fluidic channel.

In various embodiments, the method 200 is for manufacturing the microfluidic device 100 or 150 as described hereinbefore with reference to FIG. 1A or FIG. 1B, therefore, the method 200 may further include various steps correspond to providing or forming various configurations and/or components/elements of the microfluidic device 100 or 150 as described herein according to various embodiments, and thus such corresponding steps need not be repeated with respect to the method 200 for clarity and conciseness. In other words, various embodiments described herein in context of the microfluidic device 100 or 150 are analogously or correspondingly valid for the method 200 (e.g., for manufacturing the microfluidic device 100 having various configurations and/or components/elements as described herein according to various embodiments), and vice versa.

It will be appreciated by a person skilled in the art that various steps of the method 200 presented in FIG. 2 may be performed concurrently or simultaneously, rather than sequentially, as appropriate or as desired. By way of an example only, the plurality of electrode channels and the plurality of fluidic channels may be formed or fabricated in the substrate simultaneously, such as via a single-step soft-lithography.

By way of examples only and without limitation, the substrate may be formed of glass (e.g., borosilicate glass), quartz or a polymer wafer.

In various embodiments, the electrode in the electrode channel may be formed by first introducing an electrode material (in a liquid form) into the electrode channel. In this regard, for example, an original electrode material (in solid form such as an alloy wire) may be placed at or adjacent an inlet of the electrode channel (e.g., at a reservoir portion). The microfluidic device may be subjected to a thermal treatment (e.g., heated to an appropriate temperature) to melt the electrode material into a liquid form to introduce (flow) the melted electrode material via the inlet to fill the electrode channel (e.g., by virtue of capillary forces). After the electrode material has filled the electrode channel, the thermal treatment may be removed so as to allow the electrode material in the liquid form to solidify (e.g., at room temperature) to form the electrode. Each electrode in the plurality of electrode channels may be formed in the above-described manner. In this manner, the electrodes formed in the plurality of electrode channels are advantageously self-aligned with the plurality of fluidic channels, as described hereinbefore. Suitable electrode materials are known in the art and thus need not be described herein. By way of an example only and without limitation, the original electrode material may be an alloy wire having a composition of about 51% indium (In), about 32.5% bismuth (Bi) and 16.5% tin (Sn), and a melting point of about 60° C.

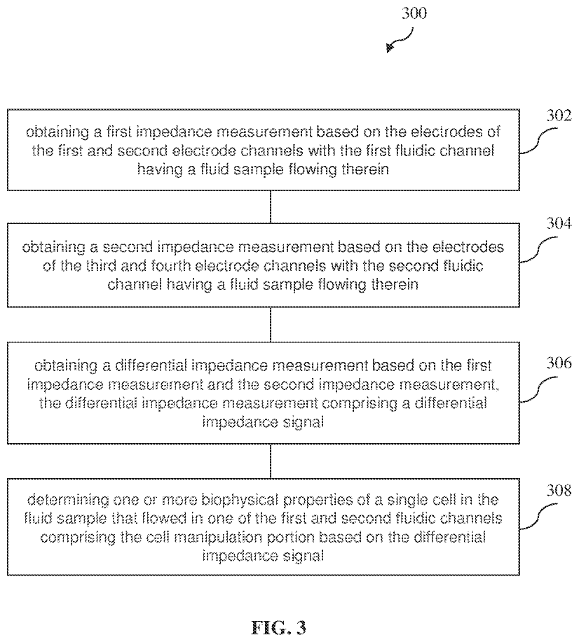

FIG. 3 depicts a schematic flow diagram of a method 300 of single cell biophysical phenotyping using the microfluidic device 100 or 150 for single cell processing as described herein with reference to FIG. 1A or FIG. 1B according to various embodiments. The method 300 comprises: obtaining (at 302) a first impedance measurement based on the electrodes of the first and second electrode channels with the first fluidic channel having a fluid sample flowing therein; obtaining (at 304) a second impedance measurement based on the electrodes of the third and fourth electrode channels with the second fluidic channel having a fluid sample flowing therein; obtaining (at 306) a differential impedance measurement based on the first impedance measurement and the second impedance measurement, the differential impedance measurement comprising a differential impedance signal; and determining (308) one or more biophysical properties of a single cell in the fluid sample that flowed in one of the first and second fluidic channels comprising the cell manipulation portion based on the differential impedance signal.

Similarly, it will be appreciated by a person skilled in the art that various steps of the method 300 presented in FIG. 3 may be performed concurrently or simultaneously, rather than sequentially, as appropriate or as desired. By way of an example only, the first impedance measurement and the second impedance measurement may be obtained simultaneously.

In various embodiments, the differential impedance measurement may be obtained based on a difference between the first impedance measurement and the second impedance measurement. In particular, the differential impedance signal may be obtained based on a first impedance signal in the first impedance measurement and a second impedance signal in the second impedance measurement.

In various embodiments, the one or more biophysical properties of the single cell is determined based on the differential impedance signal obtained at least over (or during) a time period where the single cell (only one single cell) flowed through the cell manipulation portion of the above-mentioned one of the first and second fluidic channels.

In various embodiments, over (or during) the above-mentioned time period, no cell flowed through a corresponding portion of the other one of the first and second fluidic channels. For example, in the example configuration of FIG. 1A, the above-mentioned corresponding portion may be a non-cell manipulation portion as shown in FIG. 1A, whereas in the example configuration of FIG. 1B, the corresponding portion may be the second cell manipulation portion 120b as shown in FIG. 1B.

Accordingly, during the above-mentioned time period, a pair of electrodes (e.g., the first and second electrodes) may measure an electrical impedance therebetween via the corresponding fluidic channel (e.g., the first fluidic channel 118a comprising the cell manipulation portion 120) having only one single cell flowing through the cell manipulation portion therebetween, and another pair of electrodes (e.g., the third and fourth electrodes) may measure an electrical impedance therebetween via the corresponding fluidic channel (e.g., the second fluidic channel 118b) having no cell flowing through therebetween. Accordingly, over such a time period, the electrical impedance measured between the pair of electrodes via the fluidic channel comprising the cell manipulation portion having only one single cell flowing therethrough may thus correspond to that of the fluid sample including the single cell (which may be referred to as a signal electrical impedance measurement), and the electrical impedance measured between the pair of electrodes via the fluidic channel having no cell flowing therethrough may thus corresponding to that of the fluid sample only (which may be referred to as a reference electrical impedance measurement). Accordingly, a differential electrical impedance measurement may be obtained based the signal electrical impedance measurement and the reference electrical impedance measurement, such as a difference between the signal electrical impedance measurement and the reference electrical impedance measurement. For example, such a differential electrical impedance measurement may advantageously cancel out common mode drifts caused by electrode properties or surrounding environment changes, thereby improving the measurement signal quality (e.g., improved signal-to-noise ratio).

In various embodiments, for biophysical phenotyping a single cell, a portion or a section of the differential impedance signal associated with the single cell corresponding to the above-mentioned time period is detected or identified.

In various embodiments, the differential impedance signal obtained over the above-mentioned time period comprises a plurality of impedance peaks corresponding to instances (time periods or time intervals) where the single cell flowed through the plurality of constriction portions, respectively, of the cell manipulation portion of the above-mentioned one of the first and second fluidic channels.

In various embodiments, the above-mentioned determining (at 308) one or more biophysical properties of the single cell comprises determining one or more of a deformability property, an electrical property and a relaxation property of the single cell in the fluid sample that flowed in the above-mentioned one of the first and second fluidic channels based on the plurality of impedance peaks of the differential impedance signal obtained over the above-mentioned time period.

In various embodiments, the deformability property of the single cell is determined based on a width of each of the plurality of impedance peaks of the differential impedance signal obtained over the above-mentioned time period.

In various embodiments, the electrical property of the single cell is determined based on a magnitude of each of the plurality of impedance peaks of the differential impedance signal obtained over the above-mentioned time period.

In various embodiments, the relaxation property of the single cell is determined based on a comparison between a width of a first impedance peak of the plurality of impedance peaks and a width of a subsequent impedance peak of the plurality of impedance peaks of the differential impedance signal obtained over the above-mentioned time period.

In various embodiments, the subsequent impedance peak is a last impedance peak of the plurality of impedance peaks (i.e., the latest occurring impedance peak amongst the plurality of impedance peaks) of the differential impedance signal obtained over the above-mentioned time period.

FIG. 4 depicts a schematic drawing of a system 400 for single cell biophysical phenotyping according to various embodiments of the present invention, such as corresponding to the method 300 of single cell biophysical phenotyping as described hereinbefore with respect to FIG. 3 according to various embodiments. The system 400 comprises the microfluidic device 100 or 150 for single cell processing as described hereinbefore with reference to FIG. 1A or FIG. 1B; and a computing system 402 comprising: a memory 404; and at least one processor 406 communicatively coupled to the memory 404 and the microfluidic device 100, and configured to: obtain a first impedance measurement based on the electrodes of the first and second electrode channels with the first fluidic channel having a fluid sample flowing therein; obtain a second impedance measurement based on the electrodes of the third and fourth electrode channels with the second fluidic channel having a fluid sample flowing therein; obtain a differential impedance measurement based on the first impedance measurement and the second impedance measurement, the differential impedance measurement comprising a differential impedance signal; and determine one or more biophysical properties of a single cell in the fluid sample that flowed in one of the first and second fluidic channels comprising the cell manipulation portion based on the differential impedance signal.

It will be appreciated by a person skilled in the art that the at least one processor 406 may be configured to perform the required functions or operations through set(s) of instructions (e.g., software modules) executable by the at least one processor 406 to perform the required functions or operations. Accordingly, as shown in FIG. 4, the system 400 may comprise an impedance measurement module (or circuit) 410 configured to obtain a first impedance measurement based on the electrodes of the first and second electrode channels with the first fluidic channel having a fluid sample flowing therein; obtain a second impedance measurement based on the electrodes of the third and fourth electrode channels with the second fluidic channel having a fluid sample flowing therein; and obtain a differential impedance measurement based on the first impedance measurement and the second impedance measurement; and a biophysical property determining module (or circuit) 412 configured to determine one or more biophysical properties of a single cell in the fluid sample that flowed in one of the first and second fluidic channels comprising the cell manipulation portion based on the differential impedance signal.

It will be appreciated by a person skilled in the art that the above-mentioned modules are not necessarily separate modules, and one or more modules may be realized by or implemented as one functional module (e.g., a circuit or a software program) as desired or as appropriate without deviating from the scope of the present invention. For example, the impedance measurement module 410 and the biophysical property determining module 412 may be realized (e.g., compiled together) as one executable software program (e.g., software application or simply referred to as an “app”), which for example may be stored in the memory 404 and executable by the at least one processor 406 to perform the functions/operations as described herein according to various embodiments.

In various embodiments, the computing system 402 corresponds to the method 300 of single cell biophysical phenotyping as described hereinbefore with reference to FIG. 3, therefore, various functions or operations configured to be performed by the least one processor 406 may correspond to various steps of the method 300 as described hereinbefore according to various embodiments, and thus need not be repeated with respect to the system 402 for clarity and conciseness. In other words, various embodiments described herein in context of the methods are analogously valid for the respective systems, and vice versa.

For example, in various embodiments, the memory 404 may have stored therein the impedance measurement module 410 and the biophysical property determining module 412, which respectively correspond to various steps of the method 300 as described hereinbefore according to various embodiments, which are executable by the at least one processor 406 to perform the corresponding functions/operations as described herein.

A computing system, a controller, a microcontroller or any other system providing a processing capability may be provided according to various embodiments in the present disclosure. Such a system may be taken to include one or more processors and one or more computer-readable storage mediums. For example, the computing system 402 described hereinbefore may include a processor (or controller) 406 and a computer-readable storage medium (or memory) 404 which are for example used in various processing carried out therein as described herein. A memory or computer-readable storage medium used in various embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g., a microprocessor (e.g., a Complex Instruction Set Computer (CISC) processor or a Reduced Instruction Set Computer (RISC) processor). A “circuit” may also be a processor executing software, e.g., any kind of computer program, e.g., a computer program using a virtual machine code, e.g., Java. Any other kind of implementation of the respective functions which will be described in more detail below may also be understood as a “circuit” in accordance with various alternative embodiments. Similarly, a “module” may be a portion of a system according to various embodiments in the present invention and may encompass a “circuit” as above, or may be understood to be any kind of a logic-implementing entity therefrom.

Some portions of the present disclosure are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “obtaining”, “determining” or the like, refer to the actions and processes of a computer system, or similar electronic device, that manipulates and transforms data represented as physical quantities within the computer system into other data similarly represented as physical quantities within the computer system or other information storage, transmission or display devices.

The present specification also discloses a computing system (e.g., which may also be embodied as a device or an apparatus), such as the system 402, for performing the operations/functions of the methods described herein. Such a system may be specially constructed for the required purposes, or may comprise a general purpose computer or other device selectively activated or reconfigured by a computer program stored in the computer. The algorithms presented herein are not inherently related to any particular computer or other apparatus. Various general-purpose machines may be used with computer programs in accordance with the teachings herein. Alternatively, the construction of more specialized apparatus to perform the required method steps may be appropriate.

In addition, the present specification also at least implicitly discloses a computer program or software/functional module, in that it would be apparent to the person skilled in the art that the individual steps of the methods described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention. It will be appreciated by a person skilled in the art that various modules described herein (e.g., the impedance measurement module 410 and/or the biophysical property determining module 412) may be software module(s) realized by computer program(s) or set(s) of instructions executable by a computer processor to perform the required functions, or may be hardware module(s) being functional hardware unit(s) designed to perform the required functions. It will also be appreciated that a combination of hardware and software modules may be implemented.

Furthermore, various steps of a computer program/module or method described herein may be performed in parallel rather than sequentially. Such a computer program may be stored on any computer readable medium. The computer readable medium may include storage devices such as magnetic or optical disks, memory chips, or other storage devices suitable for interfacing with a general purpose computer. The computer program when loaded and executed on such a general-purpose computer effectively results in an apparatus that implements the steps of the methods described herein.

In various embodiments, there is provided a computer program product, embodied in one or more computer-readable storage mediums (non-transitory computer-readable storage medium), comprising instructions (e.g., the impedance measurement module 410 and/or the biophysical property determining module 412) executable by one or more computer processors to perform a method 300 of single cell biophysical phenotyping, as described hereinbefore with reference to FIG. 3. Accordingly, various computer programs or modules described herein may be stored in a computer program product receivable by a system therein, such as the computing system 402 as shown in FIG. 4, for execution by at least one processor 406 of the computing system 402 to perform the required or desired functions.

The software or functional modules described herein may also be implemented as hardware modules. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. Those skilled in the art will appreciate that the software or functional module(s) described herein can also be implemented as a combination of hardware and software modules.

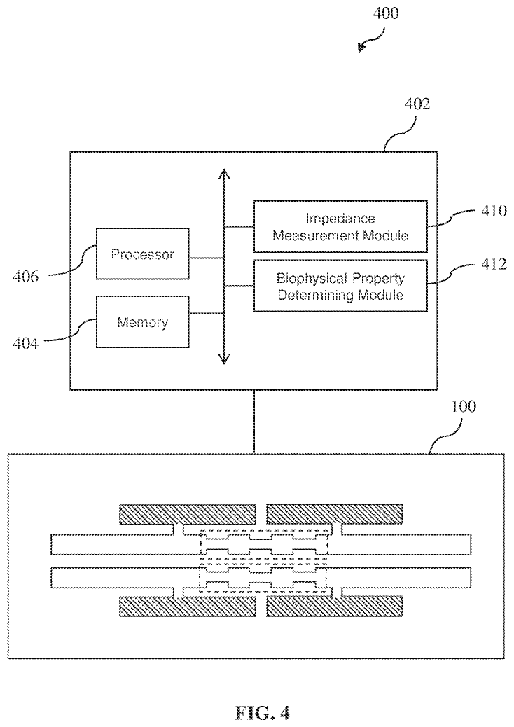

In various embodiments, the computing system 402 may be realized by any computing system (e.g., desktop or portable computing system) including at least one processor and a memory, such as a computing system 500 as schematically shown in FIG. 5 as an example only and without limitation. Various methods/steps or functional modules (e.g., the impedance measurement module 410 and/or the biophysical property determining module 412) may be implemented as software, such as a computer program being executed within the computing system 500, and instructing the computing system 500 (in particular, one or more processors therein) to conduct the methods/functions of various embodiments described herein. The computing system 500 may comprise a computer module 502, input modules, such as a keyboard 504 and a mouse 506, and a plurality of output devices such as a display 508, and a printer 510. The computer module 502 may be connected to a computer network 512 via a suitable transceiver device 514, to enable access to e.g., the Internet or other network systems such as Local Area Network (LAN) or Wide Area Network (WAN). The computer module 502 in the example may include a processor 518 for executing various instructions, a Random Access Memory (RAM) 520 and a Read Only Memory (ROM) 522. The computer module 502 may also include a number of Input/Output (I/O) interfaces, for example I/O interface 524 to the display 508, and I/O interface 526 to the keyboard 504. The components of the computer module 502 typically communicate via an interconnected bus 528 and in a manner known to the person skilled in the relevant art.

It will be appreciated by a person skilled in the art that the terminology used herein is for the purpose of describing various embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

In order that the present invention may be readily understood and put into practical effect, various example embodiments of the present invention will be described hereinafter by way of examples only and not limitations. It will be appreciated by a person skilled in the art that the present invention may, however, be embodied in various different forms or configurations and should not be construed as limited to the example embodiments set forth hereinafter. Rather, these example embodiments are provided so that this disclosure will be thorough and complete, and will fully convey the scope of the present invention to those skilled in the art.

Precise measurement of mechanical and electrical properties of single cells can yield useful information on the physiological and pathological state of cells. According to various example embodiments of the present invention, there is provided a differential multi-constriction microfluidic device (e.g., corresponding to the microfluidic device 100 or 150 as described hereinbefore according to various embodiments) with self-aligned three-dimensional (3D) electrodes to simultaneously characterize the deformability, electrical impedance and relaxation index of single cells at a high throughput manner (e.g., greater than 430 cell/min). In various example embodiments, cells are pressure-driven to flow through a series of sequential microfluidic constrictions (e.g., corresponding to the plurality of constriction portions as described hereinbefore according to various embodiments), during which deformability, electrical impedance and relaxation index (biophysical properties) of single cells are extracted simultaneously from impedance spectroscopy measurements. As will be described later below according to various example embodiments, mechanical and electrical phenotyping of untreated, Cytochalasin B treated and N-Ethylmaleimide treated MCF-7 breast cancer cells were conducted as experiments to demonstrate the ability of a classification system according to various example embodiments to distinguish different cell populations purely based on these biophysical properties. In addition, the classification of different cell types was quantified using a back propagation neural network. In this regard, the trained neural network yielded classification accuracies of 87.8% (based on only electrical impedance), 70.1% (based only on deformability), 42.7% (based only on relaxation index) and 93.3% (based on a combination of electrical impedance, deformability and relaxation index), with high sensitivity (93.3%) and specificity (93.3%) for the test group. Furthermore, various example embodiments demonstrate the cell classification of a cell mixture using the biophysical phenotyping technique according to various example embodiments with the trained neural network, which was found to be in quantitative agreement with the flow cytometric analysis using fluorescent labels. Accordingly, the concurrent electrical and mechanical phenotyping according to various example embodiments advantageously enable high-throughput and label-free single cell analysis.

In various example embodiments, the differential multi-constriction microfluidic device is configured with self-aligned 3D electrodes using low melt point alloy to simultaneously measure the deformability, electrical impedance and relaxation index of single cells, such as MCF-7 breast cancer cells, that is, biophysical phenotyping at single cell level. In various example embodiments, the total transit time and average electrical impedance of single cells passing through a multi-constriction channels were analyzed and used as indicators (or as measures) for cell deformability and electrical properties, respectively. In various example embodiments, the differential multi-constriction channel was designed to examine the cell deformation and relaxation process based on the electrical impedance measurement instead of using complex image analysis, which cannot be achieved by the conventional impedance-based deformability flow cytometry with single-constriction channels. In various example embodiments, relaxation index of single cells was determined by comparing the transit time information of cells through successive constrictions. Furthermore, in various example embodiments, a back propagation neural network is provided and applied to quantify the cell type classification based on the above-mentioned three biophysical properties measured or determined. In this regard, experimental results demonstrated that biophysical phenotyping with a combination of the three biophysical properties can significantly improve cell classification accuracy with high sensitivity and specificity, thus advantageously providing applications in single cell detection and classification.

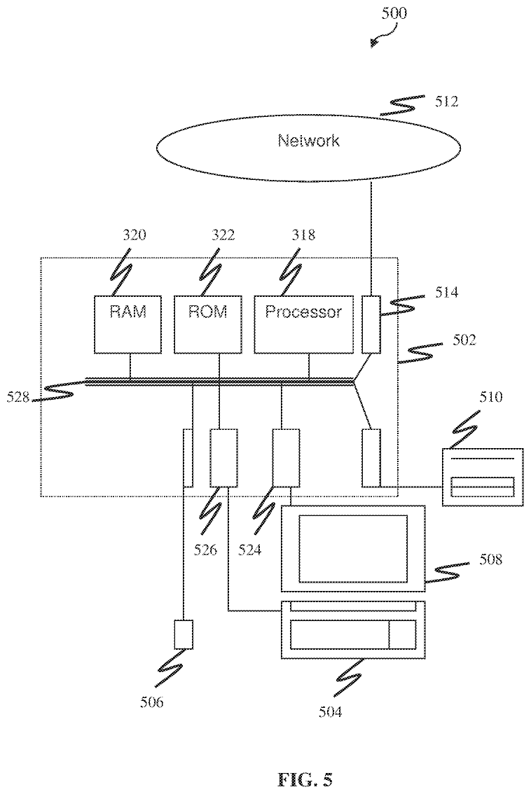

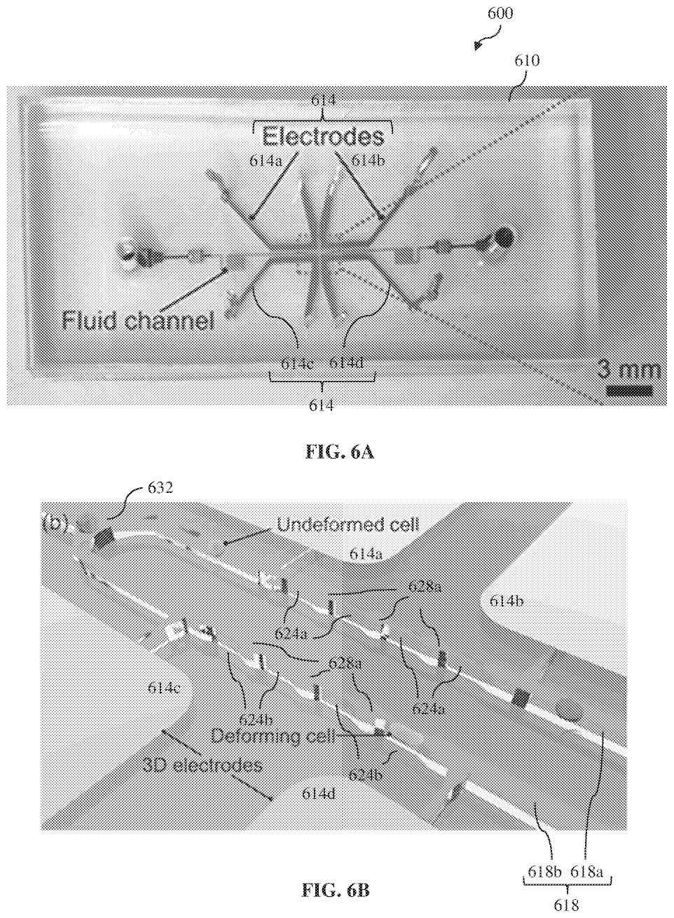

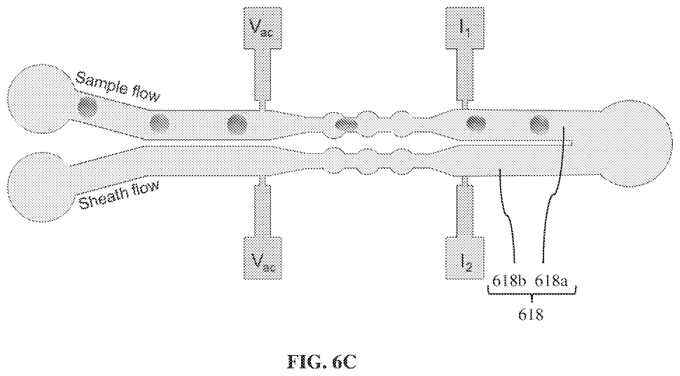

By way of an example only and without limitation, FIG. 6A depicts a top view of an example microfluidic device (microfluidic biophysical phenotyping device) 600 fabricated for single cell processing according various example embodiments of the present invention. FIG. 6B depicts an enlarged 3D schematic of the dotted boxed section of the microfluidic device 600 shown in FIG. 6A, along with illustrative single cells flowing. The microfluidic device 600 comprises: a substrate 610; a plurality of electrode channels 614, comprising a first electrode channel 614a, a second electrode channel 614b, a third electrode channel 614c and a fourth electrode channel 614d, provided in the substrate 610, each of the plurality of electrode channels 614 containing an electrode material to form an electrode; and a plurality of fluidic channels 618 (see FIG. 6B), comprising a first fluidic channel 618a and a second fluidic channel 618b, provided in the substrate 610, each of the plurality of fluidic channels 618 comprising a cell manipulation portion and is configured to form a fluid pathway for allowing a fluid sample to flow through. The cell manipulation portion comprises a plurality of constriction portions 624a/624b. The first and second electrode channels 614a/614b are each coupled to the first fluidic channel 618a and the electrodes of the first and second electrode channels 614a/614b are configured to measure an electrical impedance therebetween via the first fluidic channel 618a. The third and fourth electrode channels 614c/614d are each coupled to the second fluidic channel 618b and the electrodes of the third and fourth electrode channels 614c/614d are configured to measure an electrical impedance therebetween via the second fluidic channel 618b.

As shown in FIG. 6B, two identical fluidic channels 618a, 618b are provided for individual cells to flow through, each fluidic channel 618a/618b comprising a series of four constriction portions 624a/624b (which may simply be referred to as constrictions herein) and a series of four relaxation portions 628a/628b. By way of an example only and without limitation, each constriction portion 624a/624b may have an elongated or a rectangular shape or configuration having example dimensions of 50 pin in length, 10 μm in width and 20 μm in height, and each relaxation portion 628a/628b may have an expanded shape or configuration having example dimensions of 50 pin in length, 10 μm in width (a cross-sectional width) and 20 μm in height. The expanded configuration may be of any shape as appropriate or as desired, as long as it provides an expanded region with respect to the constriction portion for allowing each single cell to flow through unhindered, such as circular, oval, rectangular, square, diamond shape and so on. The non-cell manipulation portion of each fluidic channel 618a/618b may have cross-sectional dimensions of 10 μm in width and 20 μm in height. For example, the above-mentioned example dimensions for each fluidic channel 618a/618b may be configured (designed or sized) to process MCF-7 breast cancer cells (e.g., which may have sizes in a range of about 12 μm to about 28 μm) used in the experiments.

In the example embodiment of FIG. 6B, the fluidic channels 618 are each coupled to a flow splitter 632, via the respective input portion thereof. In this regard, the flow splitter 632 has an input portion configured to receive an input fluid sample from a fluid inlet of the microfluidic device 600 and two output portions configured to output fluid samples (divided from the input fluid sample) to the two fluidic channels 618, respectively, coupled thereto. With this configuration, the fluid samples flowing through the fluidic channels 618 are from the same input fluid sample.

However, the present invention is not limited to the configuration as shown in FIG. 6B. For example, in various example embodiments, the two fluidic channels 618 may be coupled to (in fluid communication with) two fluid inlets of the microfluidic device for receiving input fluid samples, respectively. With this configuration, the two fluid samples flowing through the two fluidic channels 618 may be the plurality of input fluid samples received, respectively. For example, as shown in FIG. 6C, the first fluidic channel 618a may be configured to receive a first input fluid sample comprising single cells desired to be analyzed and the second fluidic channel 618b may be configured to receive a second input fluid sample without any cell. The second input fluid sample may be the same as the first fluid sample except that the second fluid sample is without any cell. The first fluidic channel may then function as a signal channel and the second fluidic channel may then function as a reference channel.

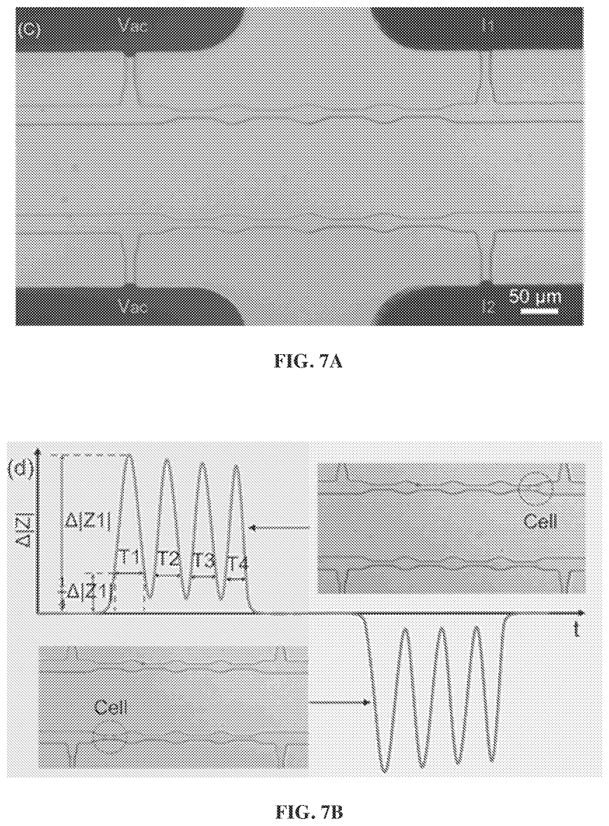

In the example microfluidic device 600, the 3D electrodes 614 are self-aligned and in direct contact with the corresponding fluidic channels (i.e., in direct contact with the sample fluid when flowing through the corresponding fluidic channel) for electrical impedance measurement. A differential electrical impedance measurement can be utilized in the experiments because of the differential channel design. FIG. 7A depicts a microscopic image of a section of the fluidic channels 618 including the cell manipulation portions, along with notations indicating the electrical measurement setup. As shown in FIG. 7A, an input voltage V_{ac} (50 KHz; 2 V) is applied to the two left electrodes, and currents are measured from the upper right (I₁) and lower right (I₂) electrodes to obtain the differential impedance (Δ|Z|). FIG. 7B depicts two example scenarios, namely, a first scenario of a single cell flowing through the upper or first fluidic channel 618a (in particular, the cell manipulation portion thereof) and a second scenario of a second cell flowing through the lower or second fluidic channel 618b (in particular, the cell manipulation portion thereof), along with the corresponding differential electrical signal profiles obtained. As shown in FIG. 7B, in the first scenario, when a single cell flows through the upper fluidic channel 618a (in particular, the cell manipulation portion thereof), the impedance of the single cell (i.e., fluid sample with the single cell) is measured by the upper two electrodes (functioning as sensing electrodes), while the lower two electrodes measure the electrical impedance of the medium (i.e., fluid sample without any cell). On the other hand, in the second scenario, the lower two electrodes (functioning as sensing electrodes) measure the cell impedance when a single cell flows through the lower fluidic channel 618b (in particular, the cell manipulation portion thereof). In this regard, when a single cell squeezes into the constriction, the less conductive cell blocks or affects the electrical field lines in the constriction, thereby leading to an increase in the electrical impedance. Based on the differential impedance measurement technique according to various example embodiments, four sequential impedance peaks can be observed above the signal baseline (i.e., positive peaks) as a cell passes through the four constrictions 624a in the upper pathway, and four sequential impedance peaks can be observed below the signal baseline (i.e., negative peaks) when a cell passes through the four constrictions 624b in the lower pathway. For example, the differential impedance measurement can cancel out any common mode drifts caused by the electrode properties or surrounding environment changes, such as the temperature and conductivity. In various example embodiments, the transit time of a single cell squeezing through each constriction 624a/624b can be determined, based on the width of each impedance peak at, for example, the ¼ peak height. As an illustration, in FIG. 7B, T1, T2, T3 and T4 denote, respectively, the transit time of the cell passing through the first, second, third and fourth constrictions 624a, and the corresponding differential impedance magnitudes are denoted as Δ|Z1|, Δ|Z3| and Δ|Z4|, respectively. According to various example embodiments, for a given cell size, the transit time of a cell passing through the constrictions 624a/624b indicates the cell deformability, and the magnitude of each impedance peak indicates the electrical properties of the cell.

A deformed cell would relax back towards its initial shape after released from the constriction. According to various example embodiments, in order to evaluate the cell deformation and relaxation process through the successive constrictions 624a/624b, the transit time of the cell through the last constriction (T4) and the first constriction (T1) are compared. For example, a ratio of T4 to T1 may be calculated and defined as the relaxation index (i.e., relaxation index=T4/T1), serving as an indicator of the cell relaxation capacity. For example, consider two opposite cases, a case where a cell relaxes back to its initial shape very fast and a case where the cell relaxes back to its initial shape very slowly. If the cell relaxes very fast (e.g., shorter than the time required to pass through the relaxation region), the cell would recover to nearly a spherical shape before entering the next constriction. Therefore, the cell enters each constriction with a nearly consistent shape and mechanical property. As a result, the time required for the cell to transit through the last constriction would be comparable with the time for the cell transiting through the first constriction. In this case, the relaxation index will be close to unity. On the other hand, if a cell relaxes very slowly, it would retain a deformed shape before entering the last constriction, and thus requires less time to transit through the last constriction, compared with the time required to transit through the first constriction. In this case, the relaxation index would be significantly (e.g., correspondingly) smaller than unity.

An example method of fabricating the example microfluidic device 600 will now be described according various example embodiments. The fluidic channels 618 and the electrode channels 614 may be fabricated simultaneously using a standard photolithography and soft-lithography process, such as a single-step standard photolithography to create the device mould and then a soft-lithography process to produce the fluidic channels 618 and the electrode channels 614. Electrode materials in the form of alloy wires (e.g., 51% In, 32.5% Bi and 16.5% Sn, melting point 60° C.) may be placed at or adjacent the inlets of the electrode channels 614. The microfluidic device 600 may be placed on a heating device (e.g., a hotplate) set to an elevated temperature, such as 80° C. After a while, the alloy wires melt into a liquid form thereby allow the melted electrode material to be introduced (flowed) into the electrode channels 614 via the inlets to fill the whole electrode channels 614 automatically by capillary forces. Subsequently, the microfluidic device 600 may be removed from the hotplate to allow the liquid electrode material in the electrode channels to solidify at room temperature. In example fabrication performed, the whole electrode molding and injection process was completed within three minutes. The inlets/outlets of the electrode channels 614 may then be sealed by PDMS. The upper part of the inserted alloy wire may be connected to a surface-mounted pin header using conductive epoxy for external circuit connection. A small amount of UV epoxy may also be deposited at the junction of the alloy wire and pin header, and then solidified under UV exposure to stabilize the electronic connection. FIG. 8 shows an image of a microfluidic device fabricated in accordance with the above-described example fabrication process.

The narrow junction microchannels (e.g., corresponding to the coupling channels described hereinbefore according to various embodiments) residing in between the electrode channels 614 and the fluidic channels 618 introduce high flow resistance and great surface tension in those regions, acting as a barrier between the two types of channels. In various example embodiments, no external force (e.g., force supplied by a syringe pump or a manual press) is applied to the electrode material at the inlets to fill the electrode channels, and instead, the melting alloy is only driven by capillary force to fill the electrode channels 614, thus, advantageously preventing the melting alloy from flowing into the fluidic channels 618. In various example embodiments, it was found that the design of narrow junction microchannels and the alloy deposition technique employed (in particular, no external force being applied) together increased the success rate of device fabrication and led to a yield of nearly 100%. Furthermore, by forming the electrodes in the above-mentioned manner, the electrodes are inherently aligned with the fluidic channels 618 and in direct contact with the sample fluid (when flowing in the fluidic channels 618), which advantageously eliminates the time-consuming and challenging alignment between electrodes and fluidic channels that were required in conventional impedance-sensing devices.

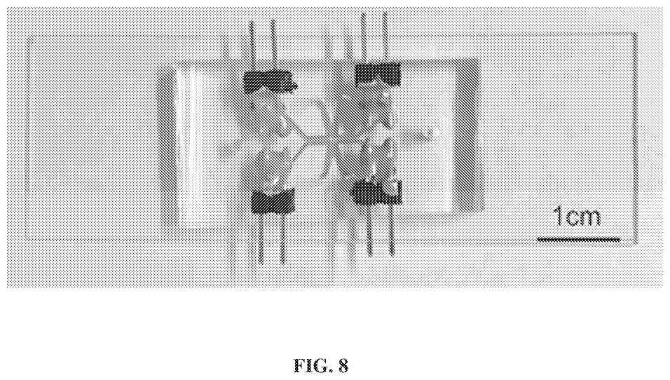

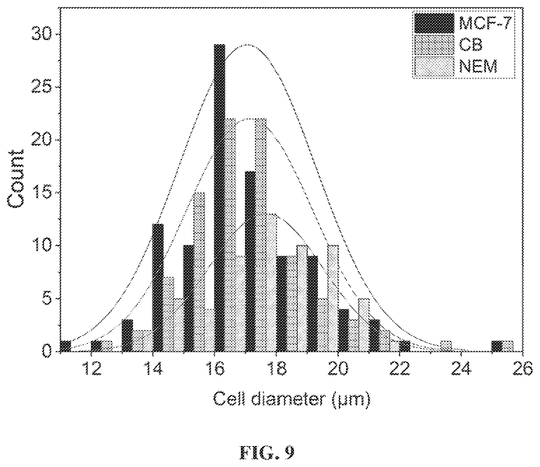

By way of an example only and without limitation, an example method of cell preparation will now be described according to various example embodiments. To ensure consistency, MCF-7 breast cancer cells from the same lot are cultured in standard conditions using the medium from the same lot. Cytochalasin B-treated MCF-7 (i.e., CB-MCF-7) and N-Ethylmaleimide-treated MCF-7 (i.e., NEM-MCF-7) may be prepared by incubating MCF-7 in 150 μM CB (Cytochalasin B) and 1 mM NEM (N-Ethylmaleimide) at 37° C. for one hour, respectively. After the treatments, cells may be spun down and resuspended in PBS for measurements. In an experiment, the above-mentioned three cell populations were prepared in accordance with the method and were found to be in a similar size range (13-22 μm) as illustrated in FIG. 9 (separately measured). In particular, FIG. 9 depicts a plot showing the cell size distributions of normal MCF-7, CB-MCF-7 and NEM-MCF-7 measured by cellSens Standard software under 20× objective lens. In order to further test the performance of the example microfluidic device 600, an experiment were performed whereby MCF-7 and NEM-MCF-7 cells were mixed together and then divided into two sample groups. Since NEM-MCF-7 cells were dead after the treatment of 1 mM NEM, propidium iodide (PI) solution (1.0 mg/ml in water) was used to stain the first sample to distinguish the MCF-7 and NEM-MCF-7 cells using flow cytometry (MACSQuant Analyzer). The nuclei staining dye PI cannot pass through a live cell membrane and therefore only NEM-MCF-7 cells were stained. The second sample was measured in the example microfluidic device 600 without staining for the comparison with flow cytometry.

The experimental setup and data analysis will now be described according to various example embodiments. The fluidic flow in the microfluidic device was controlled by a pressure control system (Fluigent MFCS-EZ). A positive pressure was applied at the fluidic inlet of the microfluidic device 600 to drive the flow. Generally, a high fluid driving pressure results in a high throughput, at a price of sacrificing the time resolution during the transit time measurement. In the experiment, 500 mbar was selected as the fluid driving pressure to balance the throughput and sensitivity. Before the cells were injected into the microfluidic channels, the microfluidic channels were filled with 1% BSA (in PBS) for 20 mins to prevent any non-specific adsorption of cells to the channel walls. Cells were then continuously introduced into the microfluidic device at the desired pressure. Electrical impedance data were recorded by an impedance spectroscope (e.g., HF2IS, Zurich Instruments) and analysed using a custom-written Matlab program.

In various example embodiments, in order to evaluate the efficiency of the classification of different cell types based on the concurrent mechanical and electrical phenotyping, a back propagation neural network (Python 3.5) including three layers was configured for pattern recognition. The whole dataset for each cell type may be divided into three parts, for example, including training data (70%), validation data (15%) and test data (15%), to quantify the cell classification accuracy (true classification/total population). FIG. 10 depicts a schematic drawing illustrating an example back propagation neural network configured for classifying cell types based on biophysical properties measured according to various example embodiments of the present invention, including three inputs, two hidden nodes and three outputs. The example back propagation neural network may include a first layer (an input layer), a middle layer (a hidden layer) and a third or final layer (an output layer). In various example embodiments, each node or neuron in the hidden and output layers may turn the sum of the inputs into an output using a sigmoid activation function y=1/(1+e^{-x}), where x is the sum of input signals moderated by link weights, and y is the output of that node. In various example embodiments, the difference (i.e., error) between the correct answer given by the training data and their actual output may be propagated backward to guide the refinement of the link weights inside the neural network. In an example experiment, four hundreds of iterations of learning were performed to train the neural network and thus link weights will converge to a configuration such that the trained neural network produces outputs that reflect the training examples. In various example embodiments, there are three groups of input parameters, namely, electrical impedance, total transit time, and relaxation index. The output results are three cell types, which is this example are MCF-7, CB-MCF-7 and NEM-MCF-7.

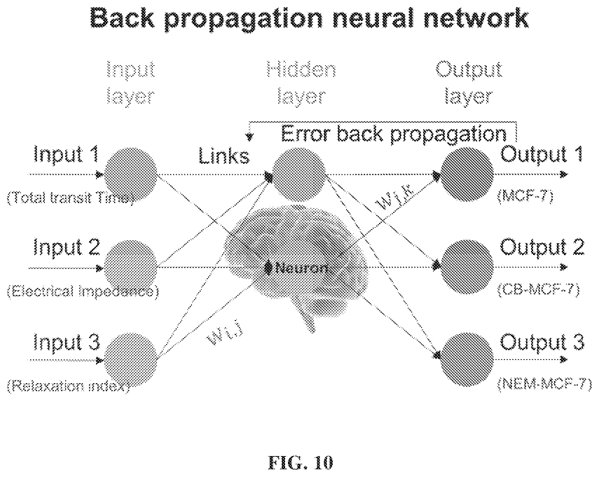

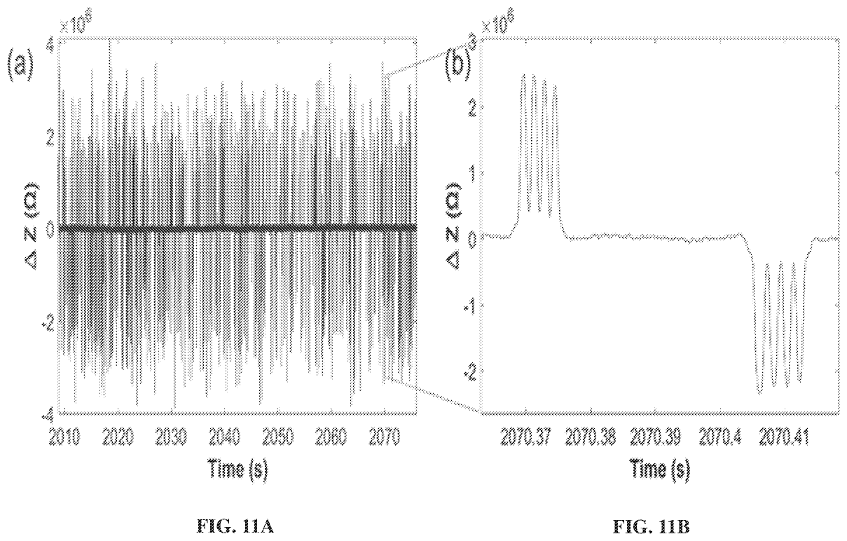

Various experimental results will now be described. In various example embodiments, the functionality of the example microfluidic device 600 was validated by characterizing the three biophysical properties of chemically treated MCF-7 cells and normal MCF-7 cells. FIG. 11A depicts a differential impedance signal of MCF-7 cells measured at 50 kHz frequency and 500 mbar flow pressure, and FIG. 11B depicts an enlarged view of a section of the differential impedance signal. FIG. 11A illustrates an impedance signal of MCF-7 cells passing through the constriction regions in the first and second fluidic channels 118, measured from one single experiment. FIG. 11B depicts an enlarged section of the impedance signal shown in FIG. 11A, which corresponds to an event where one cell transits through the first fluidic channel 118a (upper pathway), followed by another cell passing through the second fluidic channel 118b (lower pathway). As shown in FIG. 11B, where an enlarged view of the impedance signal is presented, as a single cell transits through the upper pathway, four successive impedance peaks are observed, respectively corresponding to the moments when the cell passes through the four constrictions in the upper pathway. Similarly, four impedance peaks are generated when a single cell transits through the lower pathway. It can also be observed that the peak width and magnitude decrease as cells pass through successive constrictions and undergo repeated deformation. The throughput was found to be more than 430 cells/min in the experiment, which may be further improved by using higher driving pressure to increase the flow rate, or by optimizing cell concentrations.

Deformability and electrical impedance analysis according to various example embodiments will now be described. In various example embodiments, to verify that the transit time of cells passing through a constriction can be determined by the deformability of cells, three populations of cells (normal MCF-7, CB-treated MCF-7 and NEM-treated MCF-7) were investigated using the example microfluidic device 600. Cytochalasin B (CB) is a microfilament-disrupting agent, which has been reported to increase cell deformability. N-ethylmaleimide (NEM) is a sulfhydryl-binding agent and has been found to reduce cell deformability. As shown in FIG. 9, the three populations of cells tested in the experiment exhibit similar cell size distributions. Since the transit time of single cells is also influenced by the cell size, the similar cell size distributions employed in the experiment ensure that the transit time is primarily dependent on the cell deformability rather than the cell size. FIG. 11C depict box charts illustrating the total transit time (total transit time=T1+T2+T3+T4) for cells to pass through the four constrictions. Cell numbers of MCF-7, CB-MCF-7 and NEM-MCF-7 are 336, 330 and 428, respectively. NEM-MCF-7 cells show the longest total transit time (7.4±1.8 ms) due to the NEM treating, while CB-MCF-7 cells exhibit the shortest total transit time (4.5±0.6 ms) because of the CB treating. Based on the total transit time information, it can be inferred that the NEM-MCF-7 cells are the least deformable one as these cells require the longest time to pass through constriction regions. On the other hand, CB-treated MCF-7 cells are the most deformable one. The deformability of normal MCF-7 cells sits in between NEM-treated and CB-treated MCF-7 cells. This result agrees with the findings in the literature that NEM makes cells less deformable and CB makes the cells more deformable. The total transit time among these three cell populations shows statistical significance (p<0.001), based on the Mann-Whitney test, as denoted by the label “***”.

FIG. 11D depict box charts illustrating the average electrical impedance of cells passing through four successive constrictions for the three cell populations, more particularly, the four sub-peaks (i.e., ΔZ_{average}=(ΔZ1+ΔZ2+ΔZ3+ΔZ4)/4) for all the three populations, measured at 50 kHz frequency. As an individual cell squeezes into the constriction, the cross-section of the constriction is mostly blocked by the deformed cell. In this case, the total electrical current is composed of the current through the cell membrane and intracellular contents, and the leakage current through the gap between the cell surface and channel wall. For the same cell type, to some extent, a larger cell means a smaller gap and thus larger impedance magnitude. Since the three cell populations tested in the experiment have similar sizes (e.g., see FIG. 9), the difference in their electrical impedance is mainly determined by the difference in the electrical properties of cell membranes and intracellular structures. NEM-MCF-7 cells were dead after the NEM treatment and their membranes were no longer a barrier to electrical current, meaning that the equivalent electrical conductance of cells increased after they were treated with NEM. Therefore, NEM-MCF-7 cells exhibit the lowest impedance magnitude. On the other hand, CB-MCF-7 cells present the largest impedance magnitude, which might be caused by the change in electrical properties of the cell membrane. The capacitance of the cell membrane seemed to have decreased after CB treatment, which led to an increase in the impedance magnitude, compared with the untreated MCF-7 cells. The untreated MCF-7 cells serve as the control group and their impedance magnitude resides in between NEM-treated and CB-treated MCF-7 cells. Since the sizes of these three cell populations are in a similar range, they cannot be easily discriminated or separated by size-based techniques such as inertial sorting. However, based on the characterization of their electrical properties, these three cell populations show a significant difference among them (p<0.001 based on the Mann-Whitney test), as denoted by the label “***”.

In FIGS. 11C and 11D, the numbers shown adjacent the box charts indicate the mean values of total transit time or electrical impedance.

Cell deformation and relaxation analysis through successive constrictions will now be described according to various example embodiments.

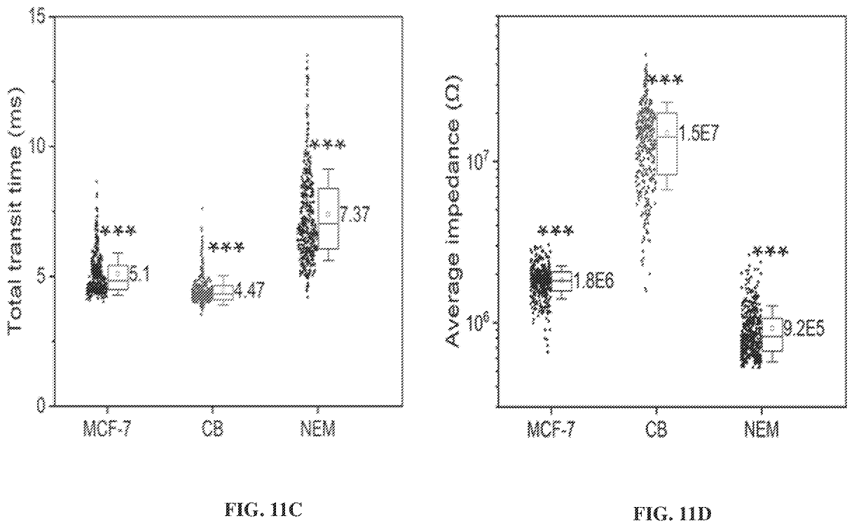

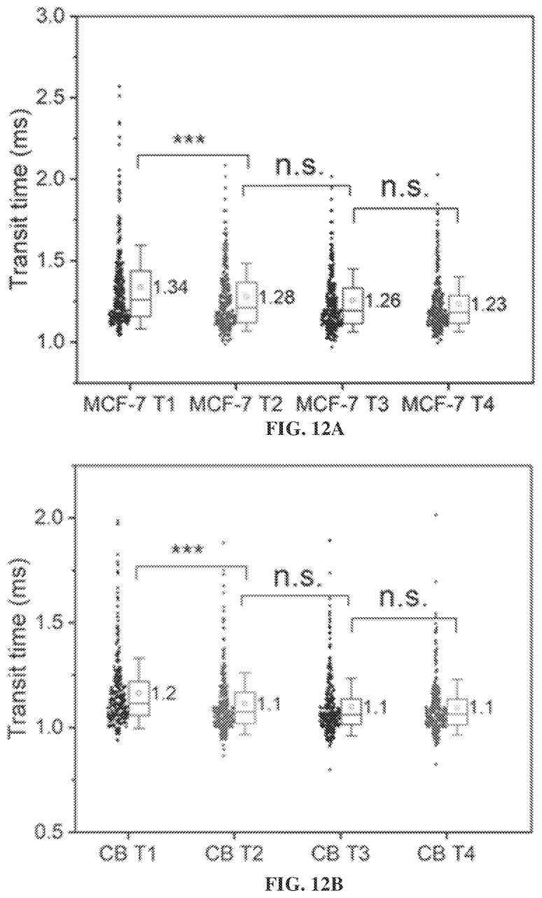

FIGS. 12A, 12B and 12C depict box charts illustrating the transit time of cells passing through each of the four successive constrictions for all the three cell populations, namely, MCF-7, CB-MCF-7 and NEM-MCF-7, respectively. Cell numbers of MCF-7, CB-MCF-7 and NEM-MCF-7 are 336, 330 and 428, respectively. The numbers shown adjacent the box charts indicate the mean values of time. The labels “***”, “*”, “*” and “n.s”. indicate a p-value of less than 0.001, 0.01, 0.05 and larger than 0.05 (statistically nonsignificant), respectively, based on the Mann-Whitney test.

For the transit time through individual constrictions, as shown in FIGS. 12A, 12B and 12C, it was found that the transit time for a single cell to pass through the first constriction is generally longer than the time needed to pass through the second, third or fourth constriction. This is because the pre-deformation facilitates the cell transit through the subsequent constrictions. At the first constriction, the cell changes from its original spherical shape into a deformed shape. After the first constriction, the cell tries to recover to its initial spherical shape in the expanded relaxation region. However, the cell relaxation capacity is dependent on the mechanical properties of the cells (i.e., viscosity and shear modulus). If the cell cannot relax fully to its initial spherical state, the cell would keep a deformed shape to some degree before entering into the next constriction. This means that, at the second constriction, the cell does not change from a spherical shape to a deformed shape, but instead changes from a less deformed shape to a more deformed shape. As a result, the transit event through the second constriction with less deformation takes a shorter time, compared with the time needed for the cell to change from its initial spherical shape into a fully deformed shape at the first constriction. Transit time comparison between the first and second constriction showed a significant difference (p<0.001, based on the Mann-Whitney test) for all the three cell populations. For the later three transit time comparison, only the NEM-MCF-7 cells exhibit the decreased significant difference, that is p<0.01 between T2 and T3 and p<0.05 between T3 and T4. There are no statistical significances (p>0.05) between T2 and T3, T3 and T4 for CB-MCF and MCF cells. This may be because, after exiting the second constriction or third constriction, NEM-MCF-7 cells are more difficult to recover to the former shape before entering into the next constriction, thereby resulting in a more deformed shape facilitating the subsequent transit events.

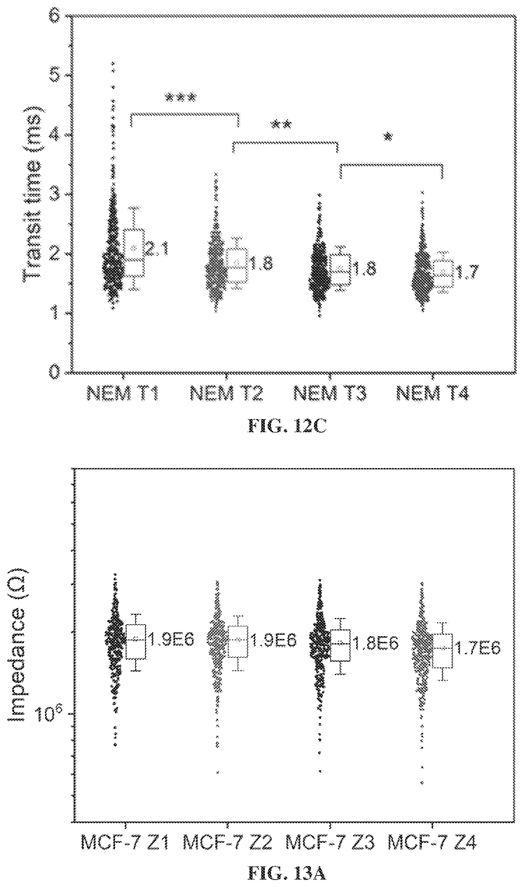

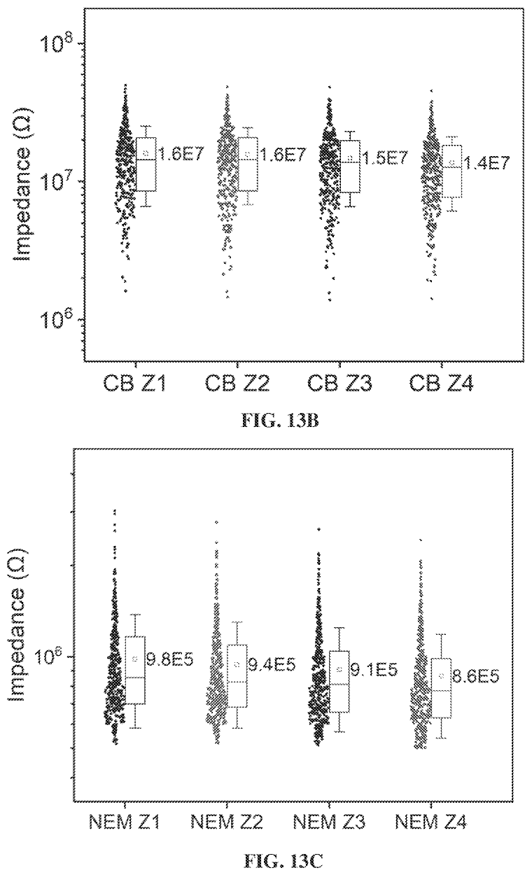

FIGS. 13A, 13B and 13C depict box charts illustrating the electrical impedance of cells passing through each of the four successive constrictions for all the three cell populations, namely, MCF-7, CB-MCF-7 and NEM-MCF-7, respectively. Cell numbers of MCF-7, CB-MCF-7 and NEM-MCF-7 are 336, 330 and 428, respectively. The numbers shown adjacent the box charts indicate the mean values of impedance. In FIGS. 13A, 13B and 13C, for all the three cell populations, a slight decline of electrical impedance is observed as cells transit through successive constrictions and undergo repeated deformation. One possible reason is that the gap between cells and channel walls increases as the cells consecutively transit through the four constrictions. The increase in the gap leads to an increase in the leakage current through the gap, thereby resulting in a decrease in the electrical impedance.

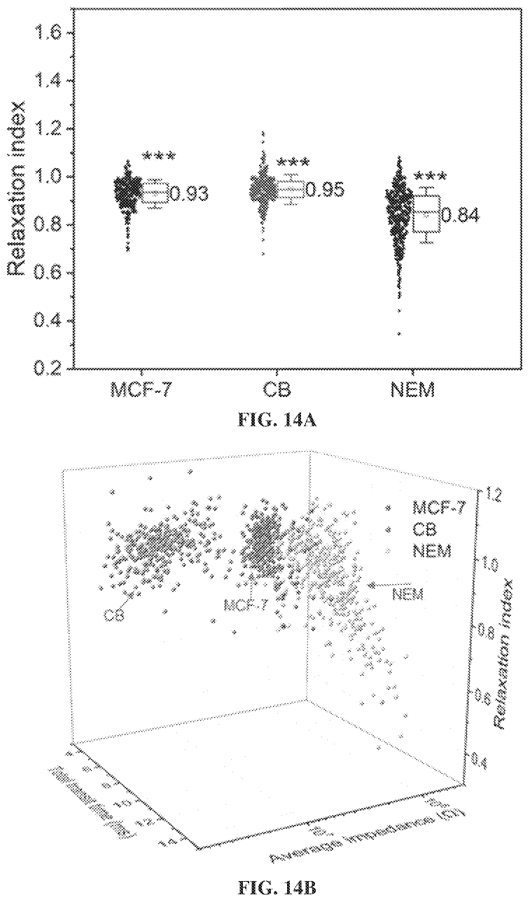

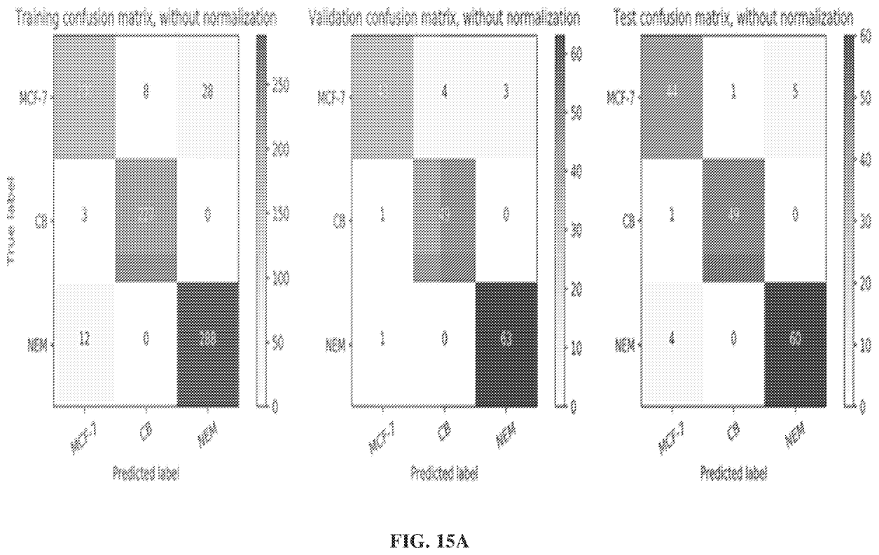

As described hereinbefore according to various example embodiments, the ratio of T4 to T1 may be defined to evaluate the cell deformation and relaxation process (i.e., relaxation index=T4/T1). A value of relaxation index closer to unity indicates that the cell relaxes faster, and vice versa. FIG. 14A depicts box charts illustrating the relaxation index, namely, the ratio of the transit time of a single cell transiting through the fourth constriction to the transit time of the cell transiting through the first constriction, and FIG. 14B depicts the 3D scatterplot of electrical impedance vs total transit time vs relaxation index. In FIG. 14B, the different cell populations can be clearly distinguished by combing the three biophysical properties (i.e., electrical impedance, total transit time and relaxation index). Cell numbers of MCF-7, CB-MCF-7 and NEM-MCF-7 are 336, 330 and 428, respectively. The numbers shown adjacent the box charts indicate the mean values of impedance. In FIG. 14A, the label “***” indicates that there is a significant difference among cell populations (p<0.001), based on the Mann-Whitney test.

As shown in FIG. 14A, a significant difference in the relaxation index can be observed among all the three cell populations (p<0.001). CB-MCF-7 cells have the highest relaxation index (0.95±0.06), meaning that these cells relax the fastest among the three cell populations. NEM-MCF-7 cells have the lowest value (0.84±0.11), indicating that these cells relax the slowest. NEM-MCF-7 cells were dead after the NEM treatment and their mechanical properties were changed as indicated by the increased total transit time. It has been reported that incomplete cell relaxation is due to an additive plastic deformation. The dead NEM-MCF-7 cells may undergo a higher proportion of plastic deformation and thus be more difficult to relax back to its original shape, resulting in the lowest relaxation index. The relaxation index evaluated according to various example embodiments demonstrates its applicability as a mechanical biomarker for single cell level biophysical phenotyping.

Accordingly, the example microfluidic device 600 enables the simultaneous characterization of the cell deformability, electrical impedance and relaxation index. In various example embodiments, these biophysical properties measured may be combined together to achieve an improved classification of different cell types. For example, as shown in FIG. 14B, three cell populations can be clearly distinguished from each other, which cannot be accomplished just based on single-domain parameter.

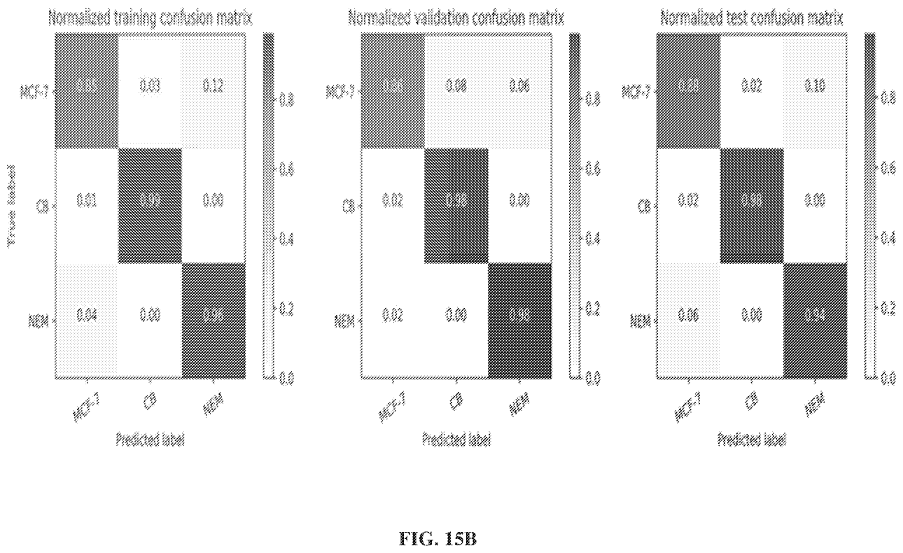

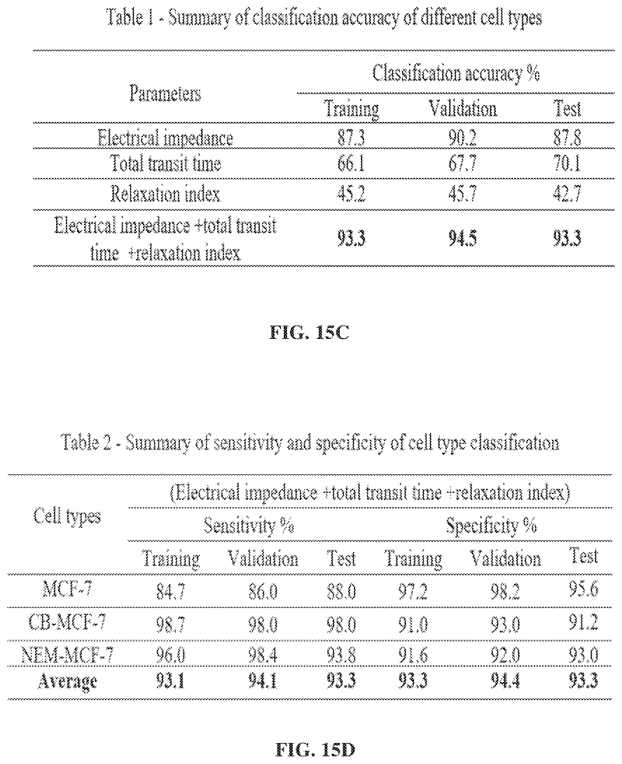

Classification of cell types based on a neural network will now be described according to various example embodiments. With the three measured biophysical properties as the inputs for the above-mentioned propagation neural network, their efficiency for the cell type classification may be quantitatively evaluated. It was found that the trained neural network achieved classification accuracies of 87.8% (based on electrical impedance), 70.1% (based on total transit time), 42.7% (based on relaxation index) and 93.3% (based on a combination of electrical impedance, total transit time and relaxation index) for the test group. Besides the classification accuracy of 93.3% (true classification/total population), the average sensitivity (true positive/(true positive+false negative)) of 93.3% and specificity (true negative/(true negative+false positive)) of 93.3% for the test group were also achieved. FIGS. 15A and 15B depict confusion matrices illustrating the efficiency of cell type classification using electrical impedance, total transit time and relaxation index. In particular, FIG. 15A depicts confusion matrices of the training, validation and test group without normalization. Values on the diagonal represent the number of cells correctly classified, while off-diagonal values represent those incorrectly classified. FIG. 15B depicts confusion matrices of the training, validation and test group with normalization to have a more visual interpretation of which cell population is misclassified.

As shown in FIG. 15A, there are more confusions between MCF-7 and NEM-MCF-7 as they have more overlaps in the measured electrical and mechanical properties. For example, in the training confusion matrix, there are 28 MCF-7 cells misclassified as NEM-MCF-7 and 12 NEM-MCF-7 cells misclassified as MCF-7. However, only 8 MCF-7 cells were misidentified as CB-MCF-7 and 3 CB-MCF-7 cells were misidentified as MCF-7. This is due to the appearance of more overlaps in the electrical impedance and mechanical properties (i.e., total transit time and relaxation index) between MCF-7 and NEM-MCF-7 cells, which can be observed from FIG. 11C, FIG. 11D and FIG. 14A. Since the number of each cell type is not equal, confusion matrix with normalization was performed to have a more visual interpretation of which cell population is misclassified. As shown in FIG. 15B, 88% percent of MCF-7, 98% percent of NEM-MCF-7 and 94% percent of NEM-MCF-7 were correctly classified for the test group. The classification accuracy, average sensitivity and specificity for the training and validation group all were higher than 93%, which are listed in Table 1 shown in FIG. 15C and Table 2 shown in FIG. 15D.

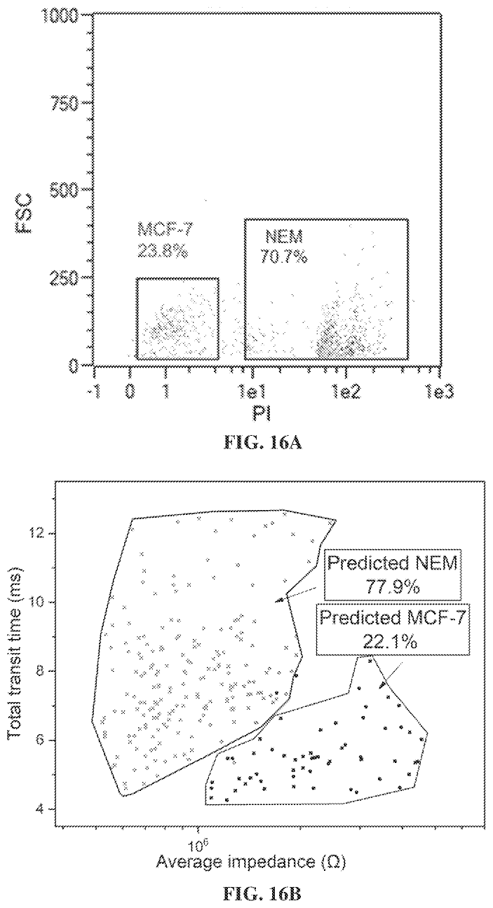

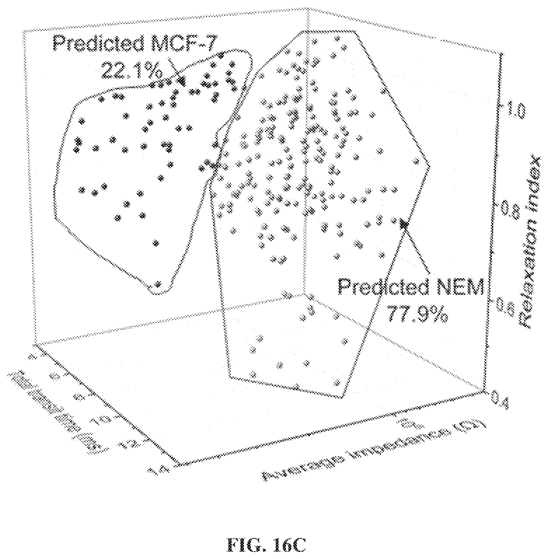

FIGS. 16A, 16B and 16C depict cell characterization using flow cytometry and the example microfluidic device 600. In particular, FIG. 16A depicts a flow cytometric scatter plot of the mixed MCF-7 and NEM-MCF-7 cells, FIG. 16B depicts a 2D scatter plot and FIG. 16C depicts a 3D scatter plot of the mixed MCF-7 and NEM-MCF-7 cells using the trained neural network with electrical impedance, total transit time and relaxation index as inputs.

The mixtures of MCF-7 and NEM-MCF-7 cells were measured using flow cytometry and the example microfluidic device 600 to further verify the performance of this biophysical phenotyping technique. As shown in FIG. 16A, using flow cytometry method, the proportion of MCF-7 and NEM-MCF-7 cells is 23.8% and 70.7%, respectively. FIG. 16B and FIG. 16C show the predicted results presenting by the trained neural network using three parameters as inputs but illustrated as 2D and 3D scatterplot, respectively. These biophysical phenotyping results of MCF-7 cells at 22.1% and NEM-MCF-7 cells at 77.9% are comparable to the flow cytometric analysis. Furthermore, fluorescent labeling process is not needed in the example microfluidic biophysical phenotyping technique according to various example embodiments as compared to flow cytometry.

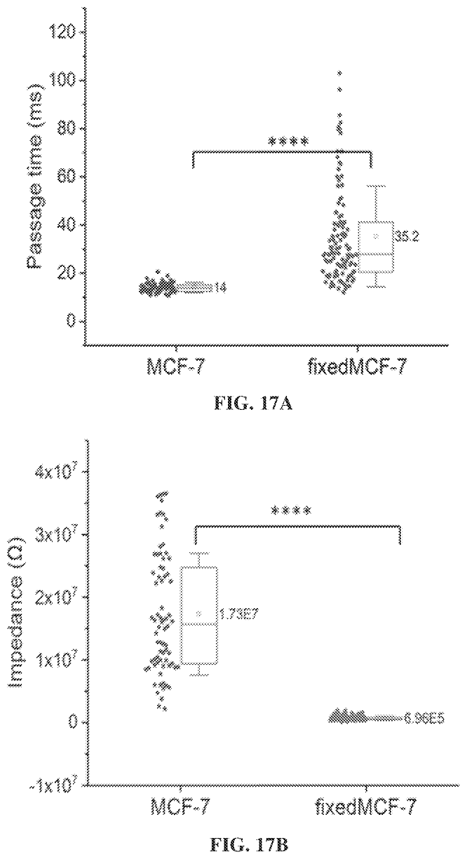

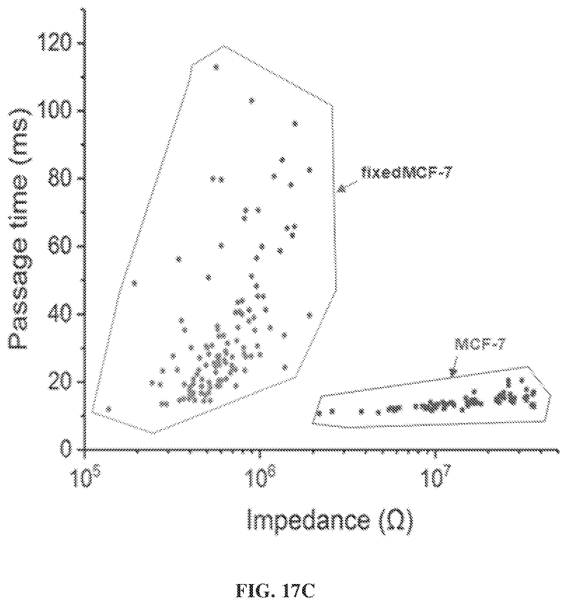

FIGS. 17A and 17B show the results of the passage time and electrical impedance, respectively, of cells passing through the constriction, measured at 200 mbar pressure. Fixed MCF-7 cells take longer to passing through the constriction, indicating fixed MCF-7 cells are less deformable than normal MCF-7 cells. The impedance magnitude of the fixed MCF-7 cells is smaller than MCF-7 cells as the cell fixation process may have heavily disrupted the cell membrane. FIG. 17C depicts a scatter plot of passage time vs impedance. Cell numbers of MCF-7 and fixed MCF-7 are 74 and 118, respectively. Label “****” indicates a p-value of less than 0.0001, based on a Mann-Whitney test. FIG. 17C shows that the use of the combination of passage time and electrical impedance can effectively distinguish different cell populations.

Accordingly, in various example embodiments, a differential multi-constriction microfluidic device with 3D electrodes is provided for high-throughput biophysical phenotyping at single cell level, referring to the simultaneous characterization of deformability (i.e., total transit time), electrical impedance and relaxation index of single cells. Compared with conventional impedance-based microfluidic device for the measurement of electrical and mechanical properties of single cells, the employment of multi-constriction channels instead of single constriction enables the evaluation of relaxation index of single cells and the method developed for the creation of self-aligned 3D electrodes greatly reduces the complexity of device fabrication. Furthermore, the differential electrical impedance approach facilitates to cancel out the environmental changes, thus improving the signal-to-noise ratio. The electrical and mechanical phenotyping of normal MCF-7 and chemical-modified MCF-7 cells were investigated according to various example embodiments and showed significant differences among them. Furthermore, according to various example embodiments, a neural network was employed for cell type classification, which quantitatively demonstrated that a combination of all of the three biophysical properties of single cells can significantly improve the classification accuracy (93.3%) of different cell populations with high sensitivity and specificity. For example, cell classification of a cell mixture using the trained neural network has achieved quantitative accuracy as compared to conventional flow cytometry. Accordingly, the microfluidic device according to various embodiments of the present invention is capable of high-throughput and label-free biophysical phenotyping of single cells, and for example, may be used as a diagnostic tool for certain diseases associated with cell mechanical or electrical properties changes. Furthermore, the simple processing of electrical signals enables real-time cell detection, further facilitating downstream cell sorting and separating processes.

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

1. A microfluidic device for single cell processing, the microfluidic device comprising:
a substrate;
a plurality of electrode channels, comprising a first electrode channel, a second electrode channel, a third electrode channel and a fourth electrode channel, provided in the substrate, each of the plurality of electrode channels containing an electrode material to form an electrode; and
a plurality of fluidic channels, comprising a first fluidic channel and a second fluidic channel, provided in the substrate, each of the plurality of fluidic channels being configured to form a fluid pathway for allowing a fluid sample to flow through and at least one of the first and second fluidic channels comprising a cell manipulation portion, the cell manipulation portion comprising a plurality of constriction portions, wherein
the first and second electrode channels are each coupled to the first fluidic channel and the electrodes of the first and second electrode channels are configured to measure an electrical impedance therebetween via the first fluidic channel, and
the third and fourth electrode channels are each coupled to the second fluidic channel and the electrodes of the third and fourth electrode channels are configured to measure an electrical impedance therebetween via the second fluidic channel.

2. The microfluidic device according to claim 1, wherein the cell manipulation portion further comprises a plurality of relaxation portions, the plurality of relaxation portions and the plurality of constriction portions in the cell manipulation portion being arranged alternately along the fluidic channel.

3. The microfluidic device according to claim 2, wherein in the cell manipulation portion, each adjacent pair of constriction portions of the plurality of constriction portions is interspaced by a corresponding relaxation portion of the plurality of relaxation portions.

4. The microfluidic device according to claim 2, wherein said at least one of the first and second fluidic channels comprises a linear portion, the linear portion comprising the cell manipulation portion.

5. The microfluidic device according to claim 2, wherein in the cell manipulation portion, each of the plurality of constriction portions has a cross-sectional dimension that is less than a cross-sectional dimension of a non-cell manipulation portion of the fluidic channel, and each of the plurality of relaxation portions has a cross-sectional dimension that is equal to or greater than the cross-sectional dimension of the non-cell manipulation portion of the fluidic channel.

6. The microfluidic device according to claim 5, wherein
said each of the plurality of constriction portions is configured to compress a single cell flowing therethrough,
said each of the plurality of relaxation portions is configured to decompress the single cell flowing therethrough, and
said single cell has a size that the microfluidic device is configured to process.

7. The microfluidic device according to claim 5, wherein
said each of the plurality of constriction portions has an elongated shape extending in a direction of the fluid pathway, and
said each of the plurality of constriction portions has dimensions which are the same.

8. The microfluidic device according to claim 1, wherein
the first and second electrode channels are coupled to the first fluidic channel at coupling portions, including a first coupling portion and a second coupling portion, of the first fluidic channel, respectively,
the third and fourth electrode channels are coupled to the second fluidic channel at coupling portions, including a third coupling portion and a fourth coupling portion, of the second fluidic channel, respectively, and
the cell manipulation portion of said at least one of the first and second fluidic channels is provided between the coupling portions of the corresponding fluidic channel such that the electrodes of the corresponding electrode channels are configured to measure the electrical impedance therebetween via the cell manipulation portion of the corresponding fluidic channel.

9. The microfluidic device according to claim 1, wherein
the first and second electrode channels are coupled to the first fluidic channel via a first coupling channel and a second coupling channel, respectively, each of the first and second coupling channels being configured to form a fluid pathway for allowing the fluid sample to flow so as to expose the electrodes of the first and second electrode channels for direct contact with the fluid sample when the fluid sample is being flowed in the first fluidic channel, and
the third and fourth electrode channels are coupled to the second fluidic channel via a third coupling channel and a fourth coupling channel, respectively, each of the third and fourth coupling channels being configured to form a fluid pathway for allowing the fluid sample to flow so as to expose the electrodes of the third and fourth electrode channels for direct contact with the fluid sample when the fluid sample is being flowed in the second fluidic channel.

10. The microfluidic device according to claim 1, wherein the cell manipulation portion comprises three or more constriction portions.

11. The microfluidic device according to claim 1, wherein
the first fluidic channel and the second fluidic channel are parallel to each other,
the first and second electrode channels each comprises an aligned portion configured to be adjacent the first fluidic channel and to be aligned with the first fluidic channel so as to extend in a parallel manner thereto; and
the third and fourth electrode channels each comprises an aligned portion configured to be adjacent the second fluidic channel and to be aligned with the second fluidic channel so as to extend in a parallel manner thereto.

12. A method of manufacturing a microfluidic device for single cell processing, the method comprising:
providing a substrate;
forming a plurality of electrode channels, comprising a first electrode channel, a second electrode channel, a third electrode channel and a fourth electrode channel, in the substrate, each of the plurality of electrode channels containing an electrode material to form an electrode; and
forming a plurality of fluidic channels, comprising a first fluidic channel and a second fluidic channel, in the substrate, each of the plurality of fluidic channels being configured to form a fluid pathway for allowing a fluid sample to flow through and at least one of the first and second fluidic channels comprising a cell manipulation portion, the cell manipulation portion comprising a plurality of constriction portions, wherein
the first and second electrode channels are each coupled to the first fluidic channel and the electrodes of the first and second electrode channels are configured to measure an electrical impedance therebetween via the first fluidic channel, and
the third and fourth electrode channels are each coupled to the second fluidic channel and the electrodes of the third and fourth electrode channels are configured to measure an electrical impedance therebetween via the second fluidic channel.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A system for single cell biophysical phenotyping, the system comprising:
a microfluidic device for single cell processing, the microfluidic device comprising:
a substrate;
a plurality of electrode channels, comprising a first electrode channel, a second electrode channel, a third electrode channel and a fourth electrode channel, provided in the substrate, each of the plurality of electrode channels containing an electrode material to form an electrode; and
a plurality of fluidic channels, comprising a first fluidic channel and a second fluidic channel, provided in the substrate, each of the plurality of fluidic channels being configured to form a fluid pathway for allowing a fluid sample to flow through and at least one of the first and second fluidic channels comprising a cell manipulation portion, the cell manipulation portion comprising a plurality of constriction portions, wherein
the first and second electrode channels are each coupled to the first fluidic channel and the electrodes of the first and second electrode channels are configured to measure an electrical impedance therebetween via the first fluidic channel, and
the third and fourth electrode channels are each coupled to the second fluidic channel and the electrodes of the third and fourth electrode channels are configured to measure an electrical impedance therebetween via the second fluidic channel; and
a computing system comprising:
a memory; and
at least one processor communicatively coupled to the memory and the microfluidic device, and configured to:
obtain a first impedance measurement based on the electrodes of the first and second electrode channels with the first fluidic channel having a fluid sample flowing therein;
obtain a second impedance measurement based on the electrodes of the third and fourth electrode channels with the second fluidic channel having a fluid sample flowing therein;
obtain a differential impedance measurement based on the first impedance measurement and the second impedance measurement, the differential impedance measurement comprising a differential impedance signal; and
determine one or more biophysical properties of a single cell in the fluid sample that flowed in one of the first and second fluidic channels comprising the cell manipulation portion based on the differential impedance signal.

33. The system according to claim 32, wherein the one or more biophysical properties of the single cell is determined based on the differential impedance signal obtained at least over a time period where the single cell flowed through the cell manipulation portion of said one of the first and second fluidic channels.

34. The system according to claim 33, wherein over said time period, no cell flowed through a corresponding portion of the other one of the first and second fluidic channels.

35. The system according to claim 33, wherein the differential impedance signal obtained over said time period comprises a plurality of impedance peaks corresponding to instances where the single cell flowed through the plurality of constriction portions, respectively, of the cell manipulation portion of said one of the first and second fluidic channels.

36. The system according to claim 35, wherein said determining one or more biophysical properties of the single cell comprises determining one or more of a deformability property, an electrical property and a relaxation property of the single cell in the fluid sample that flowed in said one of the first and second fluidic channels based on the plurality of impedance peaks of the differential impedance signal obtained over said time period.

37. The system according to claim 36, wherein the deformability property of the single cell is determined based on a width of each of the plurality of impedance peaks of the differential impedance signal obtained over said time period.

38. The system according to claim 36, wherein the electrical property of the single cell is determined based on a magnitude of each of the plurality of impedance peaks of the differential impedance signal obtained over said time period.

39. The system according to claim 36, wherein the relaxation property of the single cell is determined based on a comparison between a width of a first impedance peak of the plurality of impedance peaks and a width of a subsequent impedance peak of the plurality of impedance peaks of the differential impedance signal obtained over said time period.

40. (canceled)
